(19) United States
(12) Patent Application Publication
(10) Pub. No.: US 2009/0029997 A1
(43) Pub. Date: Jan. 29, 2009

(54) Thiazole Derivatives and Use Thereof

(75) Inventors: Anna Quattropani, Geneva (CH); Vincent Pomel, Groisy (FR); Thomas Rueckle, Geneva (CH); Tania Grippi-Vallotton, Puplinge (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: LABORATOIRES SERONO SA, Coinsins, Vaud (CH)

(21) Appl. No.: 12/159,663

(22) PCT Filed: Jan. 22, 2007
(86) PCT No.: PCT/EP2007/050618

§ 371 Date: Jun. 30, 2008

Related U.S. Application Data
(60) Provisional application No. 60/762,953, filed on Jan. 27, 2006.

(30) Foreign Application Priority Data
Jan. 23, 2006 (EP) 06100724.1

(51) Int. Cl. A61K 31/5377; C07D 417/14; C07D 401/14; A61K 31/454; A61K 31/427; A61P 35/00; A61P 17/06; A61P 19/02; A61P 25/28; A61K 31/4439; A61K 31/496; C07D 277/02

(52) U.S. Cl. 514/236.5; 548/195; 514/371; 514/254.02; 544/369; 546/270.7; 514/342; 544/133; 546/209; 514/326

(57) ABSTRACT

The present invention is related to thiazole derivatives of Formula (I) in particular for the treatment and/or prophylaxis of autoimmune disorders and/or inflammatory diseases, cardiovascular diseases, neurodegenerative diseases, bacterial or viral infections, kidney diseases, platelet aggregation, cancer, transplantation, graft rejection or lung injuries.

### FIELD OF THE INVENTION

This present invention is related to thiazole derivatives of Formula (I), pharmaceutical formulations thereof, methods of preparation thereof and to their use for the treatment and/or prophylaxis of autoimmune disorders and/or inflammatory diseases, cardiovascular diseases, neurodegenerative diseases, bacterial or viral infections, allergy, asthma, pancreatitis, multi-organ failure, kidney diseases, platelet aggregation, cancer, transplantation, sperm motility, erythrocyte deficiency, sperm motility, graft rejection or lung injuries. Specifically, the present invention is related to thiazole derivatives for the preparation of a pharmaceutical formulation for the modulation, notably the inhibition of the activity or function of the phosphoinositide-3-kinases, PI3Ks.

### BACKGROUND OF THE INVENTION

Phosphoinositide 3-kinases (PI3Ks) have a critical signalling role in cell proliferation, cell survival, vascularization, membrane trafficking, glucose transport, neurite outgrowth, membrane ruffling, superoxide production, actin reorganization and chemotaxis (Cantley, 2000, Science, 296, 1655-1657 and Vanhaesebroeck et al., 2001, Annu. Rev. Biochem., 70, 535-602).

The term PI3K is given to a family of lipid kinases which, in mammals, consists in eight identified PI3Ks that are divided into three sub-families according to their structure and their substrate specificity.

Class I group of PI3Ks consists in two sub-groups, Class IA and Class IB.

Class IA consists in a 85 kDa regulatory unit (responsible for protein-protein interactions via the interaction of Src homology 2 (SH2) domain with phosphotyrosine residues of other proteins) and a catalytic sub-unit of 110 kDa. Three catalytic forms (p100α, p110β and p110δ) and five regulatory isoforms (p85α, p85β, p55γ, p55α and p50α) exist for this class.

Class IB are stimulated by G protein βγ sub-units of heterodimeric G proteins. The only characterized member of Class IB is PI3Kγ (P110γ catalytic sub-unit complexed with a 101-kDa regulatory protein, p101).

Class II PI3Ks comprises α, β and γ isoforms, which are approximately of 170 kDa and characterized by the presence of a C-terminal C2 domain.

Class III PI3Ks includes the phosphatidylinositol specific 3-kinases.

The evolutionary conserved isoforms p110α and β are ubiquitously expressed, while δ and γ are more specifically expressed in the haematopoetic cell system, smooth muscle cells, myocytes and endothelial cells (Vanhaesebroeck et al., 1997, Trends Biochem Sci., 22(7), 267-72). Their expression might also be regulated in an inducible manner depending on the cellular, tissue type and stimuli as well as disease context.

PI3Ks are enzymes involved in phospholipid signalling and are activated in response to a variety of extra-cellular signals such as growth factors, mitogens, integrins (cell-cell interactions) hormones, cytokines, viruses and neurotransmitters and also by intracellular cross regulation by other signalling molecules (cross-talk, where the original signal can activate some parallel pathways that in a second step transmit signals to PI3Ks by intra-cellular signalling events), such as small GTPases, kinases or phosphatases for example.

Phosphatidylinositol (PtdIns) is the basic building block for the intracellular inositol lipids in eukaryotic cells, consisting of D-myo-inositol-1-phosphate (Ins1P) linked via its phosphate group to diacylglycerol. The inositol head group of PtdIns has five free hydroxy groups and three of these are found to be phosphorylated in cells in different combinations. PtdIns and its phosphorylated derivatives are collectively referred as inositol phospholipids or phosphoinositides (PIs). Eight PI species have been documented in eukaryotic cells (Vanhaesebroeck et al., 2001, above). PIs all reside in membranes and are substrates for kinases, phosphatases and lipases.

In vitro, PI3Ks phosphorylate the 3-hydroxyl group of the inositol ring in three different substrates: phosphatidylinositol (PtdIns), phosphatidylinositol-4-phosphate (PI(4)P) and phosphatidylinositol-4,5-biphosphate (PI(4,5)P₂), respectively generating three lipid products, namely phosphatidylinositol 3-monophosphate (PI(3)P), phosphatidylinositol 3,4-bisphosphate (PI(3,4)P₂) and phosphatidylinositol 3,4,5-trisphosphate (PI(3,4,5)P₃ (see Scheme A below).

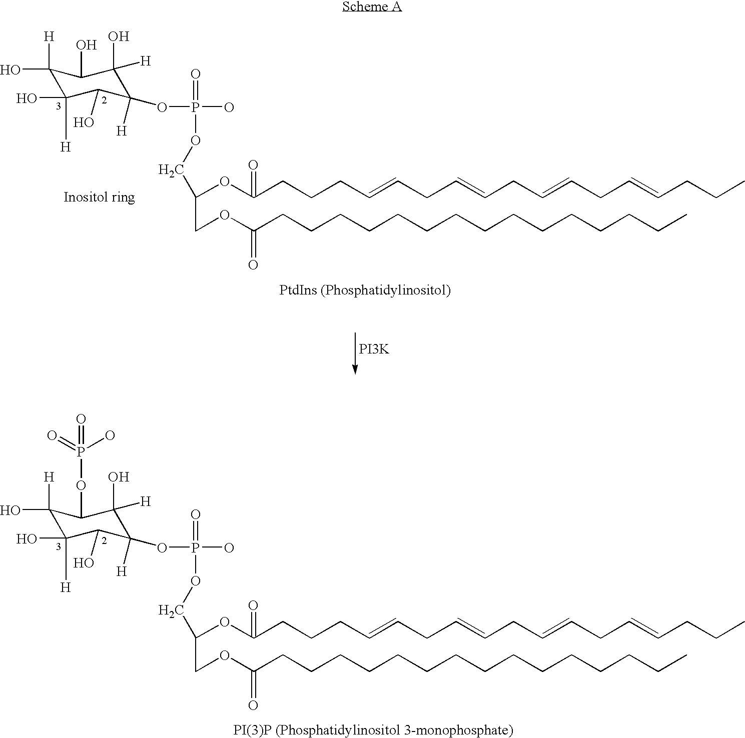

The preferred substrate for Class I PI3Ks is PI(4,5)P₂. Class II PIKs have a strong preference for PtdIns as substrate over PI(4)P and PI(4,5)P₂. Class III PI3Ks can only use PtdIns as substrate in vivo and are likely to be responsible for the generation of most PI(3)P in cells (Vanhaesebroeck et al., 2001, above).

The phosphoinositides intracellular signalling pathway begins with the binding of a signalling molecule (extracellular ligands, stimuli, receptor dimerization, transactivation by heterologous receptor (e.g. receptor tyrosine kinase)) to a G-protein linked transmembrane receptor integrated into the plasma membrane resulting in the activation of PI3Ks.

Once activated, PI3Ks convert the membrane phospholipid PI(4,5)P₂ into PI(3,4,5)P₃ which in turn can be further converted into another 3′ phosphorylated form of phosphoinositides by 5′-specific phosphoinositide phosphatases, thus PI3K enzymatic activity results either directly or indirectly in the generation of two 3′-phosphoinositide sub-types that function as second messengers in intra-cellular signal transduction (Toker et al., 2002, Cell Mol. Life. Sci. 59(5) 761-79).

The role as second messengers of phosphorylated products of PtdIns act is involved in a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Stein, 2000, Mol. Med. Today 6(9) 347-57). Chemotaxis—the directed movement of cells toward a concentration gradient of chemical attractants, also called chemokines is involved in many important diseases such as inflammation/auto-immunity, neurodegeneration, angiogenesis, invasion/metastasis and wound healing (Wyman et al., 2000, Immunol Today 21(6) 260-4; Hirsch et al., 2000, Science 287(5455) 1049-53; Hirsch et al., 2001, FASEB J 15(11) 2019-21 and Gerard et al, 2001, Nat. Immunol. 2(2) 108-15).

PI3-kinase activation, is therefore believed to be involved in a range of cellular responses including cell growth, differentiation and apoptosis (Parker et al., 1995, Current Biology, 5, 577-99; Yao et al., 1995, Science, 267, 2003-05).

Recent biochemical studies revealed that, Class I PI3Ks (e.g. Class IB isoform PI3Kγ) are dual-specific kinase enzymes, i.e. they display both lipid kinase activity (phosphorylation of phospho-inositides) as well as protein kinase activity, as they are able to induce the phosphorylation of other protein as substrates, including auto-phosphorylation as intra-molecular regulatory mechanism.

PI3Ks appear to be involved in a number of aspects of leukocyte activation. A p85-associated PI3-kinase activity has been shown to physically associate with the cytoplasmic domain of CD28, which is an important co-stimulatory molecule for the activation of T-cells in response to antigen. These effects are linked to increases in the transcription of a number of genes including interleukin-2 (IL-2), an important T cell growth factor (Fraser et al., 1991, Science, 251, 313-16). Mutation of CD28 such that it can longer interact with PI3-kinase leads to a failure to initiate IL-2 production, suggesting a critical role for PI3-kinase in T cell activation.

Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

PI3Kγ has been identified as a mediator of G beta-gamma-dependent regulation of JNK activity wherein G beta-gamma are subunits of heterotrimeric G proteins.

Recently, it has been described that PI3Kγ relays inflammatory signals through various G(i)-coupled receptors (Laffargue et al., 2002, Immunity 16(3) 441-51) and its central to mast cell function, stimuli in context of leukocytes, immunology includes cytokines, chemokines, adenosines, antibodies, integrins, aggregation factors, growth factors, viruses or hormones for example (Lawlor et al., 2001, J. Cell. Sci., 114 (Pt 16) 2903-1).

Specific inhibitors against individual members of a family of enzymes provide valuable tools for deciphering functions of each enzyme.

Two compounds, LY294002 and wortmannin (cf. hereinafter), have been widely used as PI3-kinase inhibitors. These compounds are non-specific PI3K inhibitors, as they do not distinguish among the four members of Class I PI3-kinases.

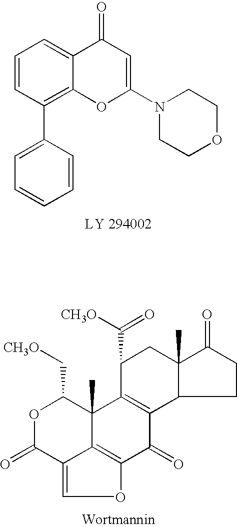

IC₅₀ values of wortmannin against each of the various Class I PI3-kinases are in the range of 1-10 nM and IC₅₀ values for LY294002 against each of these PI3-kinases are about 15-μM (Fruman et al., 1998, Ann. Rev. Biochem., 67, 481-507), also 5-10 mM on CK2 protein kinase and some inhibitory activity on phospholipases.

Wortmannin is a fungal metabolite which irreversibly inhibits PI3K activity by binding covalently to the catalytic domain of this enzyme. Inhibition of PI3K activity by wortmannin eliminates the subsequent cellular response to the extracellular factor (Thelen et al, 1994, Proc. Natl. Acad. Sci. USA, 91, 4960-64). Experiments with wortmannin, show that PI3K activity in cells of hematopoietic lineage, particularly neutrophils, monocytes, and other types of leukocytes, is involved in many of the non-memory immune response associated with acute and chronic inflammation.

Based on studies using wortmannin, there is evidence that PI3-kinase function is also required for some aspects of leukocyte signaling through G-protein coupled receptors (Thelen et al., 1994). Moreover, it has been shown that wortmannin and LY294002 block neutrophil migration and superoxide release. However, in as much as these compounds do not distinguish among the various isoforms of PI3K, it remains unclear which particular PI3K isoform or isoforms are involved in these phenomena.

Some results have indicated that PI3K inhibitors, for example, LY294002, can increase the in vivo antitumor activity of certain cytotoxic agents (e.g. Paclitaxel) (Grant, 2003, IDrugs, 6(10), 946-948).

Recently, thiazole derivatives have been recently developed as PI3K inhibitors (WO 2005/021519; WO 04/078754 and WO 04/096797).

WO 2005/021519 discloses thiazole derivatives of the following structure:

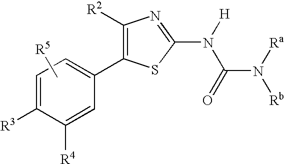

WO 04/078754 discloses thiazole derivatives of the following structure:

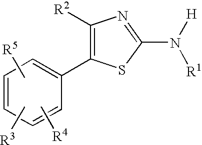

WO 04/096797 discloses thiazole derivatives of the following structure:

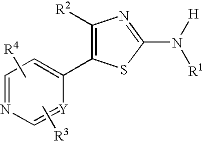

The high relevance of the PI3K pathway in some widely spread diseases stresses the need to develop inhibitors, including selective inhibitors, of PIKs.

### SUMMARY OF THE INVENTION

It is an object of the invention to provide substances which are suitable for the treatment and/or prevention of disorders related to phosphoinositide-3-kinases, PI3Ks.

It is also an object of the present invention to provide substances which are suitable for the treatment and/or prevention of auto-immune and/or inflammatory disorders.

It is also an object of the present invention to provide substances which are suitable for the treatment and/or prevention of cardiovascular diseases.

It is also an object of the present invention to provide substances which are suitable for the treatment and/or prevention of neurodegenerative disorders.

It is also an object of the present invention to provide substances which are suitable for the treatment and/or prevention of a disorder selected from bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions.

It is notably an object of the present invention to provide chemical compounds which are able to modulate, especially inhibit the activity or function of phosphoinositide-3-kinases, PI3Ks in disease states in mammals, especially in humans.

It is furthermore an object of the present invention to provide a new category of pharmaceutical formulations for the treatment of and/or diseases mediated selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions.

It is furthermore an object of the present invention to provide a method for the treatment and/or prevention of disorders selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions.

In a first aspect, the invention provides thiazole derivatives of Formula (I):

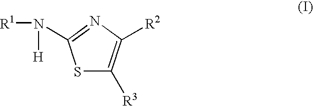

wherein R¹, R² and R³ are defined in the detailed description below.

In a second aspect, the invention provides a compound according to Formula (I) for use as a medicament.

In a third aspect, the invention provides a use of a compound according to Formula (I) for the preparation of a pharmaceutical composition for the treatment of a disorder selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions and other diseases and disorders associated with the phosphoinositide-3-kinases, PI3Ks, comprising PI3K α and γ.

In a fourth aspect, the invention provides a pharmaceutical composition comprising at least one a compound according to Formula (I) and a pharmaceutically acceptable carrier, diluent or excipient thereof.

In a fifth aspect, the invention provides a method for treating a patient suffering from a disorder selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions and other diseases and disorders associated with the phosphoinositide-3-kinases, PI3Ks. The method comprises administering a compound according to Formula (I).

In a sixth aspect, the invention provides methods of synthesis of a compound according to Formula (I).

In a seventh aspect, the invention provides compounds according to Formula (P7).

In an eighth aspect, the invention provides compounds according to Formula (P9).

### DETAILED DESCRIPTION OF THE INVENTION

The following paragraphs provide definitions of the various chemical moieties that make up the compounds according to the invention and are intended to apply uniformly through-out the specification and claims unless an otherwise expressly set out definition provides a broader definition.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like. By analogy, “C₁-C₁₂-alkyl” refers to monovalent alkyl groups having 1 to 12 carbon atoms, including “C₁-C₆-alkyl” groups and heptyl, octyl, nonyl, decanoyl, undecanoyl and dodecanoyl groups and “C₁-C₁₀-alkyl” refers to monovalent alkyl groups having 1 to 10 carbon atoms, “C₁-C₈-alkyl” refers to monovalent alkyl groups having 1 to 8 carbon atoms and “C₁-C₅-alkyl” refers to monovalent alkyl groups having 1 to 5 carbon atoms.

“Heteroalkyl” refers to C₁-C₁₂-alkyl, preferably C₁-C₆-alkyl, wherein at least one carbon has been replaced by a heteroatom selected from O, N or S, including 2-methoxy-ethyl.

“Aryl” refers to an unsaturated aromatic carbocyclic group of from 6 to 14 carbon atoms having a single ring (e.g., phenyl) or multiple condensed rings (e.g., naphthyl). Aryl include phenyl, naphthyl, phenanthrenyl and the like.

“C₁-C₆-alkyl aryl” refers to aryl groups having a C₁-C₆-alkyl substituent, including methyl phenyl, ethyl phenyl and the like.

“Aryl C₁-C₆-alkyl” refers to C₁-C₆-alkyl groups having an aryl substituent, including 3-phenylpropanoyl, benzyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to heteroaryl groups having a C₁-C₆-alkyl substituent, including methyl furyl and the like.

“Heteroaryl C₁-C₆-alkyl” refers to C₁-C₆-alkyl groups having a heteroaryl substituent, including furyl methyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to an aryl groups having a C₂-C₆-alkenyl substituent, including vinyl phenyl and the like.

“Aryl C₂-C₆-alkenyl” refers to a C₂-C₆-alkenyl groups having an aryl substituent, including phenyl vinyl and the like.

“C₂-C₆-alkenyl heteroaryl” refers to heteroaryl groups having a C₂-C₆-alkenyl substituent, including vinyl pyridinyl and the like.

“Heteroaryl C₂-C₆-alkenyl” refers to C₂-C₆-alkenyl groups having a Heteroaryl substituent, including pyridinyl vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₃-C₈-cycloalkyl” refers to a saturated carbocyclic group of from 3 to 8 carbon atoms having a single ring (e.g., cyclohexyl) or multiple condensed rings (e.g., norbornyl). C₃-C₈-cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Heterocycloalkyl include pyrrolidine, piperidine, piperazine, morpholine, tetrahydrofurane and the like.

“C₁-C₆-alkyl cycloalkyl” refers to C₃-C₈-cycloalkyl groups having a C₁-C₆-alkyl substituent, including methyl cyclopentyl and the like.

“Cycloalkyl C₁-C₆-alkyl” refers to C₁-C₆-alkyl groups having a C₃-C₈-cycloalkyl substituent, including 3-cyclopentyl propyl and the like.

“C₁-C₆-alkyl heterocycloalkyl” refers to heterocycloalkyl groups having a C₁-C₆-alkyl substituent, including 1-methylpiperazine and the like.

“Heterocycloalkyl C₁-C₆-alkyl” refers to C₁-C₆-alkyl groups having a heterocycloalkyl substituent, including 4-methyl piperidyl and the like.

“Carboxy” refers to the group —C(O)OH.

“Carboxy C₁-C₆-alkyl” refers to C₁-C₆-alkyl groups having an carboxy substituent, including 2-carboxyethyl and the like.

“Acyl” refers to the group —C(O)R where R includes H, “C₁-C₁₂-alkyl”, preferably “C₁-C₆-alkyl”, “aryl”, “heteroaryl”, “C₃-C₈-cycloalkyl”, “heterocycloalkyl”, “aryl C₁-C₆-alkyl”, “heteroaryl C₁-C₆-alkyl”, “C₃-C₈-cycloalkyl C₁-C₆-alkyl” or “heterocycloalkyl C₁-C₆-alkyl”.

“Acyl C₁-C₆-alkyl” to C₁-C₆-alkyl groups having an acyl substituent, including acetyl, 2-acetylethyl and the like.

“Acyl aryl” refers to aryl groups having an acyl substituent, including 2-acetylphenyl and the like.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Acyloxy C₁-C₆-alkyl” refers to C₁-C₆-alkyl groups having an acyloxy substituent, including propionic acid ethyl ester and the like.

“Alkoxy” refers to the group —O—R where R includes “C₁-C₆-alkyl” or “aryl” or “hetero-aryl” or “aryl C₁-C₆-alkyl” or “heteroaryl C₁-C₆-alkyl”. Preferred alkoxy groups include for example, methoxy, ethoxy, phenoxy and the like.

“Alkoxy C₁-C₆-alkyl” refers to alkoxy groups having a C₁-C₆-alkyl substituent, including methoxy, methoxyethyl and the like.

“Alkoxycarbonyl” refers to the group —C(O)OR where R includes H, “C₁-C₆-alkyl” or “aryl” or “heteroaryl” or “aryl C₁-C₆-alkyl” or “heteroaryl C₁-C₆-alkyl” or “heteroalkyl”.

“Alkoxycarbonyl C₁-C₆-alkyl” refers to C₁-C₅-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen or C₁-C₆-alkyl or aryl or heteroaryl or “aryl C₁-C₆-alkyl” or “heteroaryl C₁-C₆-alkyl”, including N-phenyl formamide.

“Aminocarbonyl C₁-C₆-alkyl” refers to C₁-C₆-alkyl groups having an aminocarbonyl substituent, including 2-(dimethylaminocarbonyl)ethyl, N-ethyl acetamide, N,N-Diethyl-acetamide and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Acylamino C₁-C₆-alkyl” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”, and where R′ and R″, together with the nitrogen atom to which they are attached, can optionally form a 3-8-membered heterocycloalkyl ring.

“Ureido C₁-C₆-alkyl” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Amino” refers to the group —NRR′ where each R, R′ is independently hydrogen or “C₁-C₆-alkyl” or “aryl” or “heteroaryl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“Amino C₁-C₆-alkyl” refers to C₁-C₅-alkyl groups having an amino substituent, including 2-(1-pyrrolidinyl)ethyl and the like.

“Ammonium” refers to a positively charged group —N⁺RR′R″, where each R,R′,R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“Ammonium C₁-C₆-alkyl” refers to C₁-C₆-alkyl groups having an ammonium substituent, including 1-ethylpyrrolidinium and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfonyloxy C₁-C₆-alkyl” refers to C₁-C₆-alkyl groups having a sulfonyloxy substituent, including 2-(methylsulfonyloxy)ethyl and the like.

“Sulfonyl” refers to group “—SO₂—R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfonyl C₁-C₆-alkyl” refers to C₁-C₅-alkyl groups having a sulfonyl substituent, including 2-(methylsulfonyl)ethyl and the like.

“Sulfinyl” refers to a group “—S(O)—R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfinyl C₁-C₆-alkyl” refers to C₁-C₆-alkyl groups having a sulfinyl substituent, including 2-(methylsulfinyl)ethyl and the like.

“Sulfanyl” refers to groups —S—R where R includes H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “alkynylheteroaryl C₂-C₆”, “cycloalkyl C₁-C₆-alkyl”, “heterocycloalkyl C₁-C₆-alkyl”. Preferred sulfanyl groups include methylsulfanyl, ethylsulfanyl, and the like.

“Sulfanyl C₁-C₆-alkyl” refers to C₁-C₅-alkyl groups having a sulfanyl substituent, including 2-(ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Sulfonylamino C₁-C₆-alkyl” refers to C₁-C₆-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “aryl C₁-C₆-alkyl” or “heteroaryl C₁-C₆-alkyl”, “aryl C₂-C₆-alkenyl”, “heteroaryl C₂-C₆-alkenyl”, “aryl C₂-C₆-alkynyl”, “heteroaryl C₂-C₆-alkynyl”, “cycloalkyl C₁-C₆-alkyl”, “heterocycloalkyl C₁-C₆-alkyl”.

“Aminosulfonyl C₁-C₆-alkyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkenyl”, “alkynyl”, “aryl”, “heteroaryl”, “cycloalkyl”, “heterocycloalkyl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like.

“Substituted” refers to groups substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “aminosulfonyl”, “ammonium”, “acyl amino”, “amino carbonyl”, “aryl”, “heteroaryl”, “sulfinyl”, “sulfonyl”, “alkoxy”, “alkoxy carbonyl”, “carbamate”, “sulfanyl”, “halogen”, trihalomethyl, cyano, hydroxy, mercapto, nitro, and the like.

“Pharmaceutically acceptable salts or complexes” refers to salts or complexes of the below-identified compounds of Formula (I) that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quarternary ammonium salt of the formula —NR,R′,R″⁺Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

Also comprised are salts formed by reaction of compounds of Formula (I) with organic or inorganic bases such as hydroxide, carbonate or bicarbonate of a metal cation such as those selected in the group consisting of alkali metals (sodium, potassium or lithium), alkaline earth metals (e.g. calcium or magnesium), or with an organic primary, secondary or tertiary alkyl amine. Amine salts derived from methyl amine, dimethylamine, trimethylamine, ethylamine, diethylamine, triethylamine, morpholine, ammonium, N-methyl-D-glucamine, N,N′-bis(phenylmethyl)-1,2-ethanediamine, tromethamine, ethanolamine, diethanolamine, ethylenediamine, N-methylmorpholine, procaine, piperidine, piperazine and the like are comtemplated being within the scope of the instant invention.

Also comprised are salts formed by reaction of compounds of Formula (I) with organic or inorganic bases such as hydroxide, carbonate or bicarbonate of a metal cation such as those selected in the group consisting of alkali metals (sodium, potassium or lithium), alkaline earth metals (e.g. calcium or magnesium), or with an organic primary, secondary or tertiary alkyl amine. Amine salts derived from methyl amine, dimethylamine, trimethylamine, ethylamine, diethylamine, triethylamine, morpholine, ammonium, N-methyl-D-glucamine, N,N′-bis(phenylmethyl)-1,2-ethanediamine, tromethamine, ethanolamine, diethanolamine, ethylenediamine, N-methylmorpholine, procaine, piperidine, piperazine and the like are comtemplated being within the scope of the instant invention.

“Pharmaceutically active derivative” refers to any compound that upon administration to the recipient, is capable of providing directly or indirectly, the activity disclosed herein. The term “indirectly” also encompasses prodrugs which may be converted to the active form of the drug via endogenous enzymes or metabolism.

It has now been found that compounds of the present invention are modulators of the Phosphatoinositide 3-kinases (PI3Ks), comprising PI3K α and γ. When the phosphatoinositide 3-kinase (PI3K) enzyme is inhibited by the compounds of the present invention, PI3K is unable to exert its enzymatic, biological and/or pharmacological effects.

The compounds of the present invention are therefore useful in the treatment and prevention of autoimmune disorders and/or inflammatory diseases, cardiovascular diseases, neurodegenerative diseases, bacterial or viral infections, allergy, asthma, pancreatitis, multi-organ failure, kidney diseases, platelet aggregation, cancer, transplantation, sperm motility, erythrocyte deficiency, graft rejection or lung injuries.

General Formula (I) according to the present invention also comprises its tautomers, its geometrical isomers, its optically active forms as enantiomers, diastereomers and its racemate forms, as well as pharmaceutically acceptable salts thereof. Preferred pharmaceutically acceptable salts of the Formula (I) are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

The compounds according to Formula (I) are suitable for the modulation, notably the inhibition of the activity of phosphatoinositide 3-kinases (PI3K). It is therefore believed that the compounds of the present invention are also particularly useful for the treatment and/or prevention of disorders, which are mediated by PI3Ks, particularly PI3K α and/or PI3K γ. Said treatment involves the modulation—notably the inhibition or the down regulation—of the phosphatoinositide 3-kinases.

The compounds according to Formula (I) are suitable for use as a medicament.

In one embodiment, the invention provides thiazole derivatives of Formula (I):

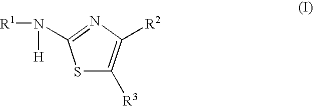

Wherein

R¹ is selected from optionally substituted aryl, optionally substituted heteroaryl, optionally substituted C₂-C₈-cycloalkyl, optionally substituted heterocycloalkyl; optionally substituted acyl, including acetyl and optionally substituted amino carbonyl such as 3-propionic acid tert-butyl ester amino carbonyl;
R² is selected from H; halogen; optionally substituted C₁-C₆-alkyl, including methyl; optionally substituted C₂-C₆-alkenyl and optionally substituted C₂-C₆-alkynyl;
R³ is selected from the following groups:

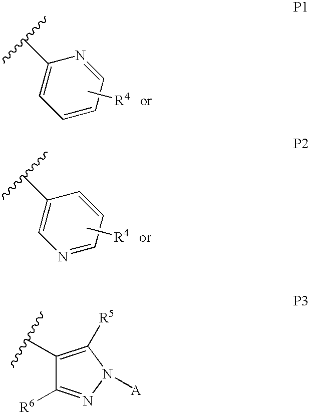

### R⁴ is —SO₂—R⁸;

R⁵ and R⁶ are independently selected from H, optionally substituted C₁-C₆-alkyl, optionally substituted C₂-C₆-alkenyl, optionally substituted C₂-C₆-alkynyl and halogen;
R⁷ is selected from H; optionally substituted C₁-C₆-alkyl, including methyl and isopropyl; optionally substituted C₂-C₆-alkenyl; optionally substituted C₂-C₆-alkynyl; optionally substituted aryl, including optionally substituted phenyl such as benzoic acid (e.g. m-benzoic acid and p-benzoic acid); optionally substituted heteroaryl, including optionally substituted pyridine such as alkyloxy pyridine (e.g. methoxy pyridine such as 6-methoxypyridin-3-yl), halogeno pyridine (e.g. chloro pyridine such as 6-chloropyridin-3-yl), heterocycloalkyl pyridine (e.g. morpholino pyridine such as 6-(morpholin-4-yl)-pyridine-3-yl); including optionally substituted imidazole such as alkyl imidazole (e.g. 1-methyl-1H-imidazol-4-yl, 2,3-dimethyl-3H-imidazol-4-yl), including optionally substituted isoxazole such as alkyl isoxazole (e.g. 3,5-dimethyl-isoxazol-4-yl), including optionally substituted pyrazole such as halogeno alkyl pyrazole (e.g. 5-chloro-1,3-dimethyl-1H-pyrazol-4-yl); optionally substituted C₃-C₈-cycloalkyl; optionally substituted heterocycloalkyl, including optionally substituted piperazine such as alkyl piperazine (e.g. 4-methyl piperazine), including optionally substituted piperidine such as N-alkoxy carbonyl piperidine (e.g. N-benzyloxycarbonyl-4-piperidine, 4-piperidine); and optionally substituted amino, including optionally substituted C₁-C₆-alkyl amine (e.g. 2-(dimethylamino)ethyl);
R⁸ is selected from optionally substituted C₁-C₆-alkyl, including methyl; optionally substituted C₂-C₆-alkenyl; optionally substituted C₂-C₆-alkynyl; optionally substituted aryl; optionally substituted heteroaryl; optionally substituted C₂-C₈-cycloalkyl, optionally substituted heterocycloalkyl and optionally substituted amino;
A is selected from H, —SO₂—R⁷; —C(O)—R⁷; optionally substituted C₁-C₆-alkyl, including methyl; optionally substituted C₂-C₆-alkenyl; optionally substituted C₂-C₆-alkynyl; optionally substituted aryl C₁-C₆-alkyl, including optionally substituted phenyl C₁-C₆-alkyl (e.g. benzyl); optionally substituted heteroaryl C₁-C₆-alkyl; optionally substituted C₂-C₈-cycloalkyl C₁-C₆-alkyl and optionally substituted heterocycloalkyl C₁-C₆-alkyl;
as well as its geometrical isomers, its optically active forms as enantiomers, diastereomers and its racemate forms, as well as pharmaceutically acceptable salts thereof.

In a specific embodiment, the invention provides thiazole derivatives of Formula (I) wherein R¹ is optionally substituted acyl.

In a specific embodiment, the invention provides thiazole derivatives of Formula (I) wherein R¹ is optionally substituted amino carbonyl.

In another specific embodiment, the invention provides thiazole derivatives of Formula (I) wherein R² is methyl.

In another specific embodiment, the invention provides thiazole derivatives of Formula (I) wherein R³ is a pyridinyl P1.

In another specific embodiment, the invention provides thiazole derivatives of Formula (I) wherein R³ is a pyridinyl P2.

In another specific embodiment, the invention provides thiazole derivatives of Formula (I) wherein R³ is a pyrazolyl P3.

In another specific embodiment, the invention provides thiazole derivatives of Formula (I) wherein R⁵ and R⁶ are H.

In another specific embodiment, the invention provides thiazole derivatives of Formula (I) wherein R⁷ is selected from H; optionally substituted C₁-C₆-alkyl, including methyl and isopropyl; optionally substituted C₂-C₆-alkenyl; optionally substituted C₂-C₆-alkynyl; optionally substituted amino, including optionally substituted C₁-C₆-alkyl amine (e.g. 2-(dimethylamino)ethyl).

In another specific embodiment, the invention provides thiazole derivatives of Formula (I) wherein R⁷ is selected from optionally substituted aryl, including optionally substituted phenyl such as benzoic acid (e.g. m-benzoic acid and p-benzoic acid); optionally substituted heteroaryl, including optionally substituted pyridine such methoxy pyridine (e.g. 6-methoxypyridin-3-yl), halogeno pyridine (e.g. chloro pyridine such as 6-chloropyridin-3-yl), morpholino pyridine (e.g. 6-(morpholin-4-yl)-pyridine-3-yl); including optionally substituted imidazole (e.g. 1-methyl-1H-imidazol-4-yl, 2,3-dimethyl-3H-imidazol-4-yl), including optionally substituted isoxazole (e.g. 3,5-dimethyl-isoxazol-4-yl), including optionally substituted pyrazole (e.g. 5-chloro-1,3-dimethyl-1H-pyrazol-4-yl); optionally substituted C₃-C₈-cycloalkyl; optionally substituted heterocycloalkyl, including optionally substituted piperazine (e.g. 4-methyl piperazine), including optionally substituted piperidine such as N-alkoxy carbonyl piperidine (e.g. N-benzyloxycarbonyl-4-piperidine, 4-piperidine) and optionally substituted amino, including optionally substituted C₁-C₆-alkyl amine (e.g. 2-(dimethylamino)ethyl);

In another specific embodiment, the invention provides thiazole derivatives of Formula (I) wherein R⁸ is selected from optionally substituted C₁-C₆-alkyl, including methyl; optionally substituted C₂-C₆-alkenyl; optionally substituted C₂-C₆-alkynyl and optionally substituted amino.

In another specific embodiment, the invention provides thiazole derivatives of Formula (I) wherein R⁸ is selected from optionally substituted aryl; optionally substituted heteroaryl; optionally substituted C₂-C₈-cycloalkyl and optionally substituted heterocycloalkyl.

In a further specific embodiment, the invention provides thiazole derivatives of Formula (I) wherein R¹ is optionally substituted acyl; R² is methyl; R⁵ and R⁶ are H; A, R³, R⁴, R⁷, R⁸ are as defined in the description.

Compounds of the present invention include in particular those of the group consisting of:

| Example | |
|---|---|
| N° | Name |
| 1 | N-[4-methyl-5-(1-methyl-1H-pyrazol-4-yl)-1,3-thiazol-2-yl]acetamide; |
| 2 | N-{4-methyl-5-[1-(methylsulfonyl)-1H-pyrazol-4-yl]-1,3-thiazol-2-yl} |
| | acetamide; |
| 3 | N-[4-methyl-5-(1H-pyrazol-4-yl)-1,3-thiazol-2-yl]acetamide; |
| 4 | N-[5-(1-benzyl-1H-pyrazol-4-yl)-4-methyl-1,3-thiazol-2-yl]acetamide; |
| 5 | 4-[2-(acetylamino)-4-methyl-1,3-thiazol-5-yl]-N-[2-(dimethylamino)ethyl]-1H- |
| | pyrazole-1-carboxamide; |
| 6 | N-(4-methyl-5-{1-[(4-methylpiperazin-1-yl)carbonyl]-1H-pyrazol-4-yl}-1,3- |
| | thiazol-2-yl)acetamide; |
| 7 | N-(5-{1-[(6-methoxypyridin-3-yl)sulfonyl]-1H-pyrazol-4-yl}-4-methyl-1,3- |
| | thiazol-2-yl)acetamide; |
| 8 | N-(4-methyl-5-{1-[(1-methyl-1H-imidazol-4-yl)sulfonyl]-1H-pyrazol-4-yl}- |
| | 1,3-thiazol-2-yl)acetamide. |
| 9 | N-(5-{1-[(6-chloropyridin-3-yl)sulfonyl]-1H-pyrazol-4-yl}-4-methyl-1,3- |
| | thiazol-2-yl)acetamide; |
| 10 | N-(4-methyl-5-{1-[(6-morpholin-4-ylpyridin-3-yl)sulfonyl]-1H-pyrazol-4-yl}- |
| | 1,3-thiazol-2-yl)acetamide; |
| 11 | N-(5-{1-[(3,5-dimethylisoxazol-4-yl)sulfonyl]-1H-pyrazol-4-yl}-4-methyl-1,3- |
| | thiazol-2-yl)acetamide; |
| 12 | N-(5-{1-[(5-chloro-1,3-dimethyl-1H-pyrazol-4-yl)sulfonyl]-1H-pyrazol-4-yl}- |
| | 4-methyl-1,3-thiazol-2-yl)acetamide; |
| 13 | N-(5-{1-[(1,2-dimethyl-1H-imidazol-5-yl)sulfonyl]-1H-pyrazol-4-yl}-4-methyl- |
| | 1,3-thiazol-2-yl)acetamide; |
| 14 | 4-({4-[2-(acetylamino)-4-methyl-1,3-thiazol-5-yl]-1H-pyrazol-1-yl}sulfonyl) |
| | benzoic acid; |
| 15 | 3-({4-[2-(acetylamino)-4-methyl-1,3-thiazol-5-yl]-1H-pyrazol-1-yl}sulfonyl) |
| | benzoic acid; |
| 16 | benzyl 4-({4-[2-(acetylamino)-4-methyl-1,3-thiazol-5-yl]-1H-pyrazol-1-yl} |
| | sulfonyl)piperidine-1-carboxylate |
| 17 | N-{5-[1-(isopropylsulfonyl)-1H-pyrazol-4-yl]-4-methyl-1,3-thiazol-2- |
| | yl}acetamide; |
| 18 | tert-butyl N-[({4-methyl-5-[1-(methylsulfonyl)-1H-pyrazol-4-yl]-1,3-thiazol-2- |
| | yl}amino)carbonyl]-beta-alaninate; |
| 19 | N-{4-methyl-5-[5-(methylsulfonyl)pyridin-2-yl]-1,3-thiazol-2-yl} acetamide. |

The compounds of the present invention are useful as medicaments. They may be used for the preparation of a medicament for the prophylaxis and/or treatment of autoimmune disorders and/or inflammatory diseases, cardiovascular diseases, neurodegenerative diseases, bacterial or viral infections, kidney diseases, platelet aggregation, cancer, transplantation, erythrocyte deficiency, graft rejection or lung injuries.

In one embodiment, the compounds of Formula (I) are useful for the treatment and/or prophylaxis of autoimmune diseases or inflammatory diseases such as multiple sclerosis, psoriasis, rheumatoid arthritis, systemic lupus erythematosis, inflammatory bowel disease, lung inflammation, thrombosis or brain infection/inflammation such as meningitis or encephalitis.

In another embodiment, the compounds of Formula (I) are useful for the treatment and/or prophylaxis of neurodegenerative diseases including Alzheimer's disease, Huntington's disease, CNS trauma, stroke or ischemic conditions.

In still a further embodiment according to the invention, the compounds of Formula (I) are useful for the treatment and/or prophylaxis of cardiovascular diseases such as atherosclerosis, heart hypertrophy, cardiac myocyte dysfunction, elevated blood pressure or vasoconstriction.

In still a further embodiment according to the invention, the compounds of Formula (I) are useful for the treatment and/or prophylaxis of erythrocyte deficiency such as an anaemia, including haemolytic anaemia, aplastic anaemia and pure red cell anaemia.

In still another embodiment according to the invention, the compounds of Formula (I) are useful for the treatment and/or prophylaxis of chronic obstructive pulmonary disease, anaphylactic shock fibrosis, psoriasis, allergic diseases, asthma, stroke or ischemic conditions, ischemia-reperfusion, platelets aggregation/activation, skeletal muscle atrophy/hypertrophy, leukocyte recruitment in cancer tissue, angiogenesis, invasion metastisis, in particular melanoma, Karposi's sarcoma, acute and chronic bacterial and viral infections, sepsis, transplantation, graft rejection, glomerulo sclerosis, glomerulo nephritis, progressive renal fibrosis, endothelial and epithelial injuries in the lung or in general lung airways inflammation.

In another embodiment according to the invention, is provided a process for the preparation of a thiazole derivative according to Formula (I), comprising the step of reacting a compound of Formula (P4) with a compound of Formula (P5) in presence of Pd and a base

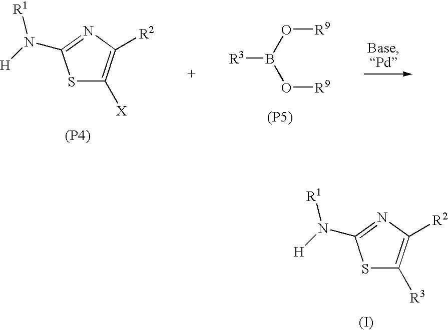

wherein R¹, R², R³ and X are as described above and wherein R⁹ is selected from H and optionally substituted C₁-C₆ alkyl.

In another embodiment according to the invention, is provided a process for the preparation of a thiazole derivative according to Formula (I), comprising the step of reacting a compound of Formula (P4) with a tin reagent of Formula (P6), in presence of Pd

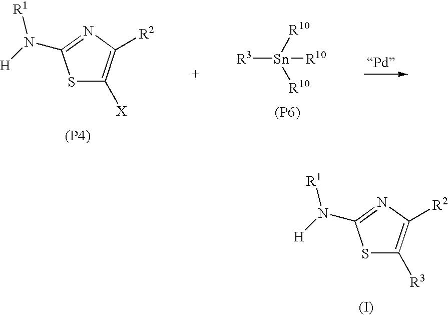

wherein R¹, R², R³, R⁹ and X are as described above and wherein R¹⁰ is selected from methyl and n-butyl.

In another embodiment, according to the invention, is provided a process for the preparation of a thiazole derivative according to Formula (I) wherein R³ is (P3) and A is COR⁷, comprising the step of reacting a compound of Formula (Ia) with a compound of Formula R⁷COCl in presence of a base such as tertiary amine, TEA, DIEA or pyridine or comprising the step of reacting a compound of Formula (Ia) with a compound of Formula R⁷COOH in presence of a coupling agent such as DCC, EDC, HOBT or PyBOP

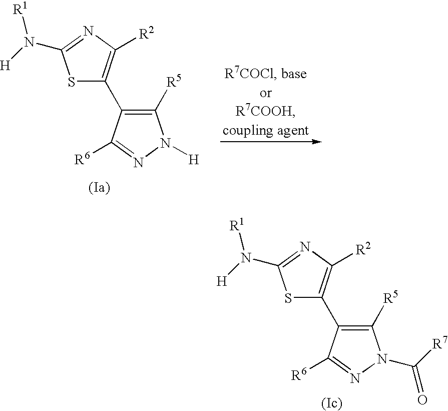

wherein R¹, R², R⁵, R⁶ and R⁷ are as defined above.

In another embodiment, according to the invention, is provided a process for the preparation of a thiazole derivative according to Formula (I) wherein R³ is (P3) and A is SO₂R⁷, comprising the step of reacting a compound of Formula (Ia) with a compound of formula R⁷COCl in presence of a base

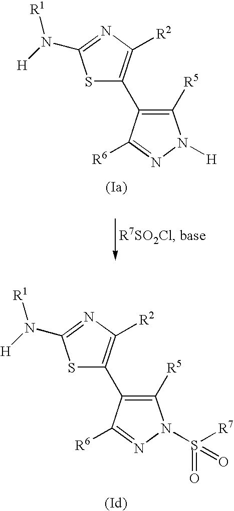

wherein R¹, R², R⁵, R⁶ and R⁷ are as defined above.

In another embodiment, according to the invention, is provided a process for the preparation of a thiazole derivative according to Formula (I) wherein R³ is (P3) and A is SO₂R⁷ comprising the step of reacting a compound of Formula (Ia) with a compound of formula AY in presence of a base such as NaH, KOH or NaOH

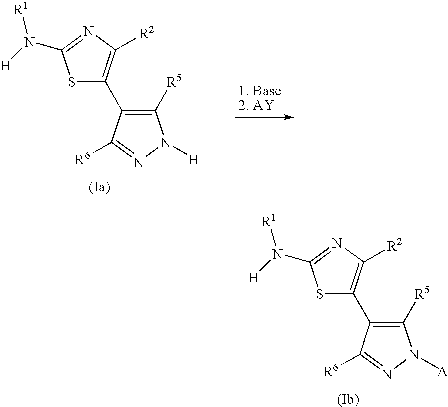

wherein R¹, R², R⁵, R⁶ and R⁷ are as defined above and Y is a leaving group such as Br or I.

In another embodiment, according to the invention, is provided a process for the preparation of a thiazole derivative according to Formula (I) wherein R³ is selected from (P1) and (P2), comprising the step of reacting a compound of Formula (P8) with a compound of Formula R⁸H in presence of a chlorination agent such as PCl₅, POCl₃ or SOCl₂

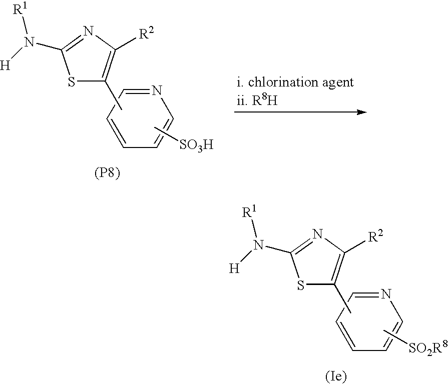

wherein R¹ and R² are as defined above and R⁸ is an optionally substituted amino group.

In another embodiment, according to the invention, is provided a process for the preparation of a thiazole derivative according to Formula (I) wherein R³ is selected from (P1) and (P2), comprising the step of reacting a compound of Formula (P9) in oxidative conditions

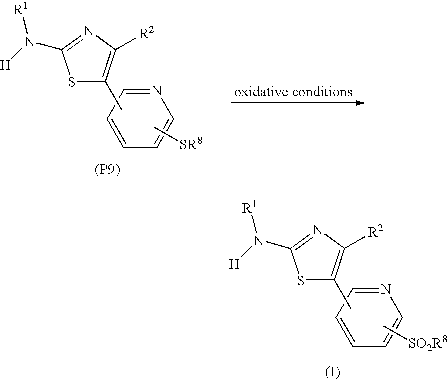

wherein R¹ and R² are as defined above and R⁸ is selected from optionally substituted C₁-C₆-alkyl, including methyl, optionally substituted C₂-C₆-alkenyl, optionally substituted C₂-C₆-alkynyl, optionally substituted aryl; optionally substituted heteroaryl, optionally substituted C₂-C₈-cycloalkyl and optionally substituted heterocycloalkyl.

In a further embodiment according to the invention, is provided a compound according to Formula (P8):

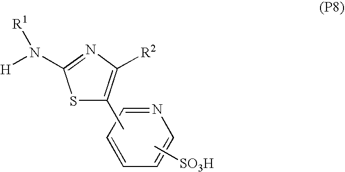

wherein R¹ and R² are as defined in the description.

In a further embodiment according to the invention, is provided a compound according to Formula (P8), selected from the following group:
5-[2-(acetylamino)-4-methyl-1,3-thiazol-5-yl]pyridine-2-sulfonic acid;
6-[2-(acetylamino)-4-methyl-1,3-thiazol-5-yl]pyridine-2-sulfonic acid;
5-[2-(acetylamino)-4-methyl-1,3-thiazol-5-yl]pyridine-3-sulfonic acid;
2-[2-(acetylamino)-4-methyl-1,3-thiazol-5-yl]pyridine-4-sulfonic acid; and
6-[2-(acetylamino)-4-methyl-1,3-thiazol-5-yl]pyridine-3-sulfonic acid.

In a further embodiment according to the invention, is provided a compound according to Formula (P9):

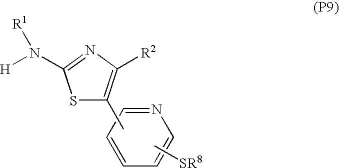

wherein R¹ and R² are as defined in the description and R⁸ is selected from optionally substituted C₁-C₆-alkyl, including methyl, optionally substituted C₂-C₆-alkenyl and optionally substituted C₂-C₆-alkynyl.

In a further embodiment according to the invention, is provided a compound according to Formula (P9), selected from the following group:
N-{4-methyl-5-[6-(methylthio)pyridin-3-yl]-1,3-thiazol-2-yl}acetamide;
N-{4-methyl-5-[6-(methylthio)pyridin-2-yl]-1,3-thiazol-2-yl}acetamide;
N-{4-methyl-5-[5-(methylthio)pyridin-3-yl]-1,3-thiazol-2-yl}acetamide;
N-{4-methyl-5-[4-(methylthio)pyridin-2-yl]-1,3-thiazol-2-yl}acetamide; and
N-{4-methyl-5-[5-(methylthio)pyridin-2-yl]-1,3-thiazol-2-yl}acetamide.

The thiazole derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

When employed as pharmaceuticals, the compounds of the present invention are typically administered in the form of a pharmaceutical composition. Hence, pharmaceutical compositions comprising a compound of Formula (I) and a pharmaceutically acceptable carrier, diluent or excipient are therefore also within the scope of the present invention. A person skilled in the art is aware of a whole variety of such carrier, diluent or excipient compounds suitable to formulate a pharmaceutical composition.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

Pharmaceutical compositions containing thiazole derivatives of this invention can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the thiazole derivative is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As above mentioned, the thiazole derivatives of Formula (I) in such compositions is typically a minor component, frequently ranging between 0.05 to 10% by weight with the remainder being the injectable carrier and the like.

The above-described components for orally administered or injectable compositions are merely representative. Further materials as well as processing techniques and the like are set out in Part 5 of Remington's Pharmaceutical Sciences, 20th Edition, 2000, Marck Publishing Company, Easton, Pa., which is incorporated herein by reference. The compounds of this invention can also be administered in sustained release forms or from sustained release drug delivery systems. A description of representative sustained release materials can also be found in the incorporated materials in Remington's Pharmaceutical Sciences.

### Synthesis of Compounds of the Invention:

The novel thiazole derivatives according to Formula (I) can be prepared from readily available starting materials by several synthetic approaches, using both solution-phase and solid-phase chemistry protocols (Kodomari et al., 2002, Tetrahedron Lett., 43, 1717-1720), either by conventional methods or by microwave-assisted techniques. Examples of synthetic pathways for the will be described.

The following abbreviations refer respectively to the definitions below:

Å (Angström), cm (centimeter), eq. (equivalent), h (hour), g (gram), M (molar), MHz (Megahertz), μl (microliter), min (minute), mg (milligram), ml (milliliter), mm (millimeter), mmol (millimole), mM (millimolar), nm (nanometer), rt (room temperature), BSA (Bovine Serum Albumin), CDI (N,N′-carbonyldiimidazole), CMC (Carboxymethyl Cellulose), DCC (dicyclohexyl carbodiimide), DCM (dichloromethane), DIEA (diisopropyl ethylamine), DMF (dimethyl formamide), DMSO (Dimethyl Sulfoxide), EDC (1-(3-dimethylaminopropyl)-3-ethyl-carbodiimidehydro-chloride), HOBt (1-hydroxybenzotriazole), HPLC (High Performance Liquid Chromatography), IHC (immunohistochemistry), Ins1P (D-myo-inositol-1-phosphate), LC (Liquid chromatography), MS (mass spectrometry), NBS (N-bromo succinimide), NIS (N-iodo succinimide), NMR (Nuclear Magnetic Resonance), PBS (Phosphate Buffered Saline), Pd(dppf)Cl₂ ([1,1′-bis(diphenylphosphino) ferrocene]palladium(II) chloride complex), PIs (Phosphoinositides), PI3Ks (Phosphoinositide 3-kinases), PI(3)P (Phosphatidylinositol 3-monophosphate), PI(3,4)P₂ (Phosphatidylinositol 3,4-bisphosphate), PI(3,4,5)P₃ (Phosphatidylinositol 3,4,5-trisphosphate), PI(4)P (Phosphatidylinositol-4-phosphate), PI(4,5)P₂) (Phosphatidyl inositol-4,5-biphosphate), PtdIns (Phosphatidylinositol), PyBOP (Benzotriazol-1-yloxy)tripyrrolidino-phosphonium hexafluorophosphate), SPA (Scintillation Proximity Assay), TEA (triethylamine), TFA (trifluoroacetic acid), THF (tetrahydrofuran), TLC (Thin Layer Chromatography), UV (Ultraviolet).

The thiazole derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

In the process illustrated in the following schemes A, X, R¹, R², R³, R⁴, R⁵, R⁶, R⁷, R⁸, R⁹ and R¹⁰ are as above-defined in the description.

The pharmaceutically acceptable cationic salts of compounds of the present invention are readily prepared by reacting the acid forms with an appropriate base, usually one equivalent, in a co-solvent. Typical bases are sodium hydroxide, sodium methoxide, sodium ethoxide, sodium hydride, potassium hydroxide, potassium methoxide, magnesium hydroxide, calcium hydroxide, benzathine, choline, diethanolamine, ethylenediamine, meglumine, benethamine, diethylamine, piperazine and tromethamine. The salt is isolated by concentration to dryness or by addition of a non-solvent. In some cases, salts can be prepared by mixing a solution of the acid with a solution of the cation (sodium ethylhexanoate, magnesium oleate), employing a solvent in which the desired cationic salt precipitates, or can be otherwise isolated by concentration and addition of a non-solvent.

The pharmaceutically acceptable anionic salt of compounds of the present invention are readily prepared by reacting the basic forms with an appropriate acid, usually on equivalent, in co-solvent. Typical acids are inorganic acids, such as hydrochloric acid, hydrobromic acid, sulfuric acid, phosphoric acid, nitric acid, and the like, or organic acids such as acetic acid, oxalic acid, tartaric acid, succinic acid, malic acid, fumaric acid, maleic acid, ascorbic acid, benzoic acid, tannic acid, palmoic acid, alginic acid, polyglutamic acid, naphthalene sulfonic acid, naphthalene disulfonic acid, and poly-galacturonic acid. The resulting salts are isolated by concentration to dryness or by addition of a non-solvent.

### Methods for Preparing Intermediates of Compounds of Formula (I).

Depending on the nature of A, R¹, R², R³, R⁴, R⁵, R⁶, R⁷ and R⁸ different synthetic strategies may be selected for the synthesis of compounds of Formula (I).

Compounds of Formula (I) may be obtained by metal catalysed cross-coupling reaction. For instance, they may be obtained by Suzuki coupling reaction between an aryl halide (P4), where X may be Br, I etc., and a boronic acid or ester (P5), where R⁹ may be H, for boronic acid derivatives, or any alkyl or substituted C₁-C₆ alkyl groups for boronic ester derivatives, including optionally —B(OR⁹)₂ forming a cycle such as boronic acid pinacol ester (Scheme 1 below) (Bellina et al., 2004, Synthesis, 2419).

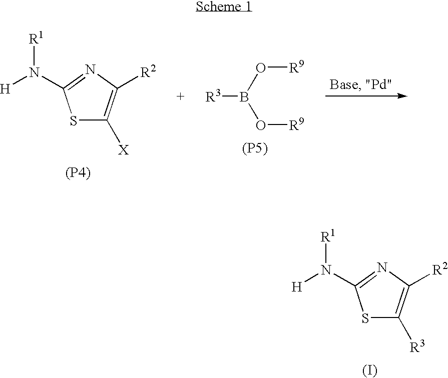

Different palladium complexes may be used, such as Pd(PPh₃)₄, [1,1′-bis(diphenyl phosphino)ferrocene]palladium(II) chloride (Pd(dppf)Cl₂), PdCl₂(PPh₃)₂, Pd(OAc)₂, with the possible addition of phosphine ligands such as PPh₃, 2-dicyclohexylphosphino-2′,6′-dimethoxy-1,1′-biphenyl. Different organic or inorganic bases may be used, such as TEA, DIEA, sodium alcoholate, such as NaOMe or NaOEt, KF, K₃PO₄ anhydrous or monohydrate, or any carbonate salts, such as K₂CO₃, Na₂CO₃, Cs₂CO₃. The solvent or solvents mixture may be selected between THF, Toluene, Dioxane, MeOH, MeCN, DMF, water, etc. The choice of solvent or solvents mixture may depend on the nature of the base, (P4) and (P5). The resulting reaction mixture may be heated, under inert atmosphere, at different temperatures, with the possible use of microwave action. All the different combinations described above may be used.

Stille coupling may be used for the preparation of compounds of Formula (I), involving the reaction between an aryl halide (P4), where X may be Br, I etc, and a tin reagent (P6), where R¹⁰ is methyl or n-butyl (Scheme 2, below) (Fugami et al., 2002, Topics in Current Chemistry, 219, 87-130). This reaction may be catalysed by different palladium complexes, such as Pd(PPh₃)₄, [1,1′-bis(diphenylphosphino)ferrocene]palladium(II) chloride (Pd(dppf) Cl₂), PdCl₂(PPh₃)₂, Pd(OAc)₂, with the possible addition of phosphine ligands, such as PPh₃, and chlorine salts, such as LiCl or ZnCl₂.

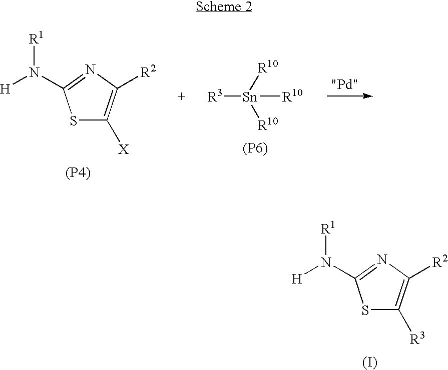

If the above set of metal catalysed cross-coupling reaction conditions is not applicable to obtain compounds according to Formula (I), suitable methods of preparation known by a person skilled in the art should be used.

Compounds of Formula (I) can be converted to alternative compounds of Formula (I), employing suitable interconversion techniques well known by a person skilled in the art.

Compounds of Formula (Ia), i.e. of Formula (I) where R³ is (P3) and A is H, may react further with any electrophile on the free NH-pyrazole (Scheme 3 below). For example, reaction of (Ia) with a base, such as NaH, KOH or NaOH, followed by the addition of an electrophile AY, wherein Y is a leaving group such as Br or I, would afford compound of Formula (Ib), wherein A may be optionally substituted C₁-C₆-alkyl, including methyl; optionally substituted C₂-C₆-alkenyl; optionally substituted C₂-C₆-alkynyl; optionally substituted aryl C₁-C₆-alkyl, including optionally substituted phenyl C₁-C₆-alkyl (e.g. benzyl); optionally substituted heteroaryl C₁-C₆-alkyl; optionally substituted C₂-C₈-cycloalkyl C₁-C₆-alkyl and optionally substituted heterocycloalkyl C₁-C₆-alkyl and Y any type of leaving group, such as Br, I etc.

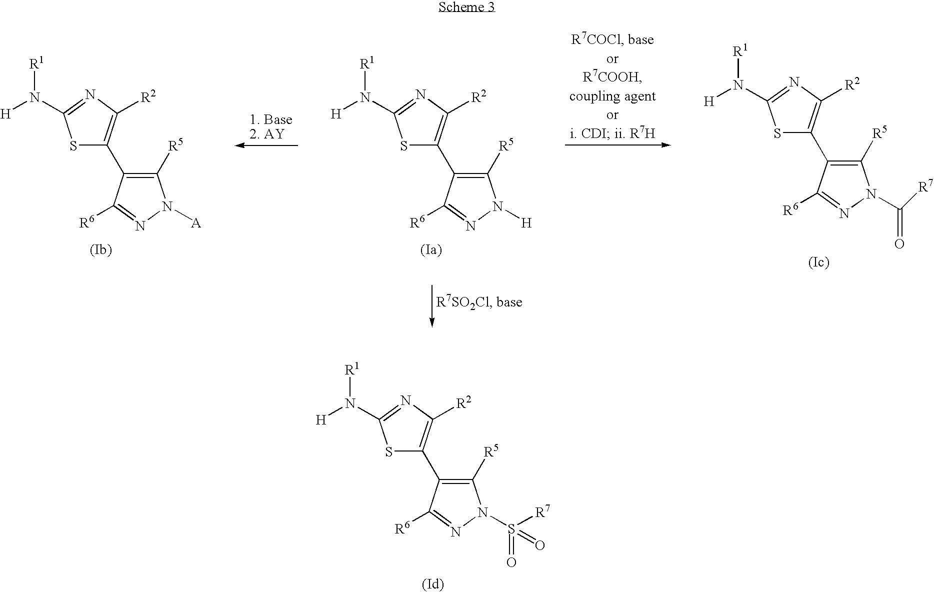

Compounds of Formula (Ic), i.e. of Formula (I) where R³ is (P3) and wherein A=COR⁷, where R⁷ is selected as defined above, may be obtained by reacting compounds of Formula (Ia) with acyl chloride R⁷COCl in the presence of a base such as a tertiary amine, TEA, DIEA or pyridine (Scheme 3 above). Acyl chlorides R⁷COCl may be commercially available or prepared from the corresponding carboxylic acid R⁷COOH under conditions known by a person skilled in the art. Compounds of Formula (Ia) may also react with carboxylic acid R⁷COOH in the presence of an activating agent, such as DCC, EDC, HOBT, PyBOP, etc. Addition of a base, such as TEA or DIEA, may be needed, depending on the nature of the coupling agent. When R⁷ is substituted amino, including optionally substituted C₁-C₆-alkyl amine (e.g. 2-(dimethylamino)ethyl amine), including optionally substituted heterocycloalkyl, including optionally substituted piperazine (e.g. 4-methyl piperazine), compounds of Formula (Ic) may be obtained by reaction of (Ia) with an amine carbonyl chloride R⁷COCl or with CDI followed by an amine R⁷H. These reactions may be achieved in the presence of a base, e.g. a tertiary amine such as TEA or DIEA.

Reactions of (Ia) with sulfonyl chloride R⁷SO₂Cl in the presence of a base, e.g. TEA, DIEA or pyridine, may afford compounds of Formula (Id), i.e. of Formula (I) where R³ is (P3) and wherein A=SO₂R⁷, where R⁷ is selected as defined above (Scheme 3 above). Solvents may be chosen between DCM, DMF, pyridine or a mixture of these solvents. Sulfonyl chlorides R⁷SO₂Cl may be commercially available or prepared by treatment of the corresponding sulfonic acids R⁷SO₂OH or their salts with chlorination agents (PCl₅, POCl₃ or SOCl₂) under standard procedures known by a person skilled in the art. Alternatively, sulfonic acid, R⁷SO₂OH, wherein R⁷ is selected as defined above, may be prepared from the corresponding alkyl bromide R⁷Br, with sodium sulfite and tetrabutylammonium iodide (Matter et al., 2002, Biorg. Med. Chem., 10, 3529-3544). It may also be prepared from the corresponding alkyl alcohol R⁷OH, starting with an esterification with thiolacetic acid, diethyl azodicarboxylate (DEAD), and triphenyl phosphine under Misunobu conditions. The resulting alkyl thioacetates of formula R⁷SAc may be oxidized with performic acid, formed in situ by mixing formic acid and hydrogen peroxide (Scheme 4), to produce the targeted sulfonic acid R⁷SO₂OH (Xu et al., 2003, Synthesis, 276-282).

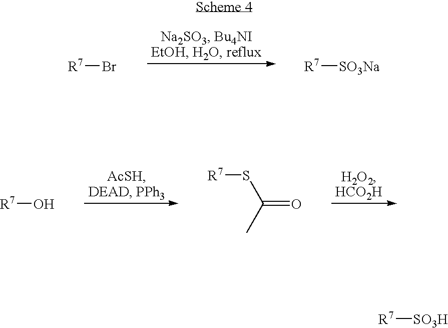

Compounds of Formula (Ie) or (If), i.e. of Formula (I) where R³ is P1 or P2, respectively and R⁸ is optionally substituted amino group, as defined above, may be obtained from intermediates (P7a) or (P7b) respectively (Scheme 5 below).

Halogen metal exchange with nBuLi in a solvent such as THF or Et₂O, at low temperature, typically −78° C., followed by addition of SO₂ as electrophile, would afford (P8a) or (P8b) respectively. These sulfonic acids (P8a) or (P8b) may be converted into their corresponding sulphonamide (Ie) or (If) respectively, where R⁸ is selected from substituted amino group, as defined above. This transformation may be achieved in two steps, sulfonyl chloride formation with chlorination agents (PCl₅, POCl₃ or SOCl₂), followed by addition of an amine, R⁸H as defined above, affording (Ie) or (If) respectively.

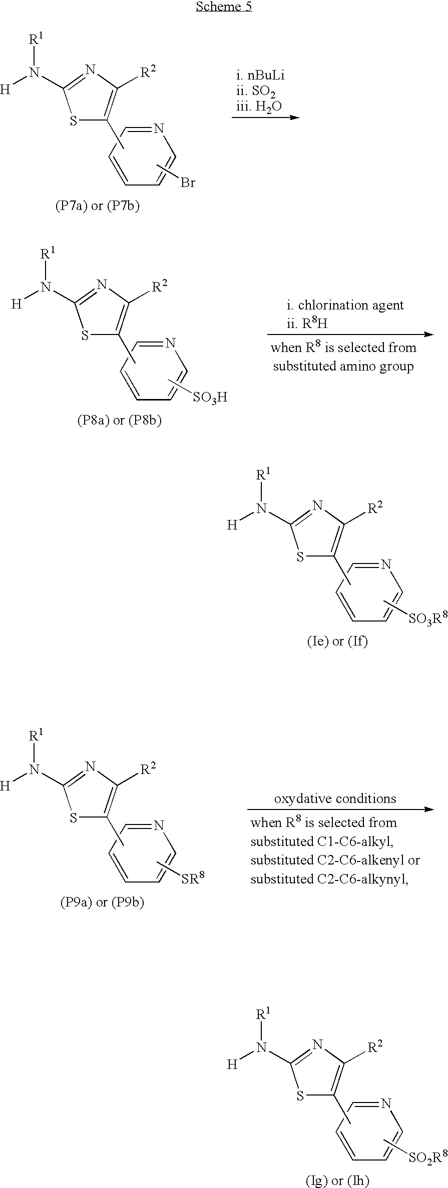

Compounds of Formula (Ig) or (Ih), i.e. of Formula (I) where R³ is P1 or P2, respectively and R⁸ is selected from optionally substituted C₁-C₆-alkyl, including methyl, optionally substituted C₂-C₆-alkenyl, optionally substituted C₂-C₆-alkynyl, optionally substituted aryl, optionally substituted heteroaryl, optionally substituted C₂-C₈-cycloalkyl and optionally substituted heterocycloalkyl as defined above, may be obtained from intermediates (P9a) or (P9b) respectively. These substituted sulfurs may be oxidized into (Ig) or (Ih) respectively, using conditions known by a person skilled in the art such as m-chloroperbenzoic acid (m-CPBA), OXONE®, dimethyldioxirane (DDO), etc.

Intermediates (P7a, b), (P8a, b) and (P9a, b) may be obtained by metal catalysed cross-coupling reaction between (P4) and the suitable boronic acid or ester for a Suzuki coupling or between (P4) and the suitable tin reagent for a Stille coupling reaction.

Compounds of Formula (Ia) to (Ih) may be obtained directly from a metal catalysed cross-coupling reaction, performing the reaction between (P4) and the suitable substituted heterocycle (P5) or (P6), as defined above.

Boronic acid or ester (P5) may be commercially available from various sources or synthesized, as it will be detailed below in the examples, using conditions known by a person skilled in the art. A boronic acid (P5a), i.e. a boronic acid of Formula (P5) wherein R⁹ is H, may be transformed into the corresponding boronic ester (P5), by heating (P5a) in the presence of an alcohol or a diol (Scheme 6 below). Boronic ester (P5) may be transformed into alternative boronic ester, using conditions known by a person skilled in the art.

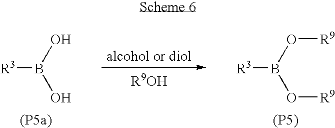

Pinacol boronic ester (P5) may be prepared by a metal coupling reaction between the corresponding heterocycle halide, (P10), where X═Br, I, etc, and bis(pinacolato)diboron (P11) (e.g. Ferrali et al., 2004, Tetrahedron Lett., 45, 5271-5274) or pinacol borane (P12) (Murata et al., 2000, J. Org. Chem., 65, 164-168) (Scheme 8 below). This reaction may be catalyzed by different palladium complexes may be used, such as Pd(PPh₃)₄, [1,1′-bis(diphenylphosphino)ferrocene]palladium(II) chloride (Pd(dppf)Cl₂), PdCl₂(PPh₃)₂, Pd(OAc)₂, with the possible addition of phosphine ligands such as PPh₃.

Different organic or inorganic bases may be used, such as TEA, DIEA, KF, KOH, or any carbonate salts, such as K₂CO₃, Na₂CO₃, Cs₂CO₃. The solvent or solvents mixture may be selected between THF, Toluene, Dioxane, MeOH, MeCN, DMF, water, etc. The resulting reaction mixture may be heated, under inert atmosphere, at different temperatures, with the possible use of microwave action.

All the different combinations described above may be used.

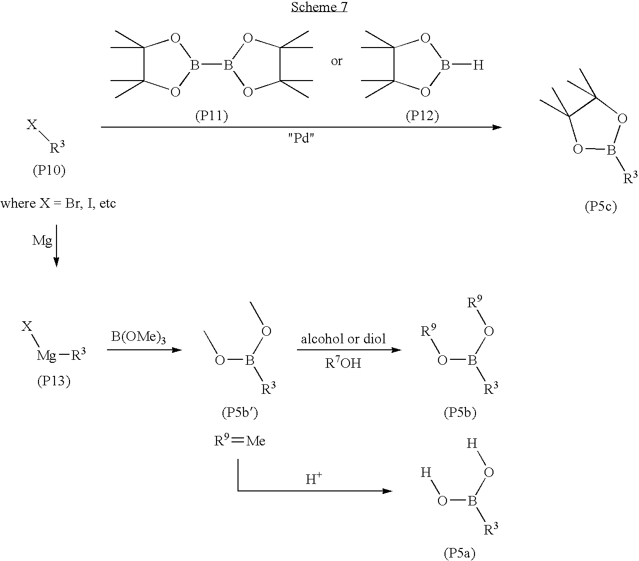

Heterocycle halide, (P10), may be first transformed into the corresponding heterocycle Grignard reagent (P13), which may react with trialkylborate, e.g. B(OMe)₃, followed either by an acidic work-up to afford the corresponding boronic acid (P5a) or by a treatment with a suitable alcohol or diol R⁹OH to afford the corresponding boronic ester (P5) (Iwong et al., 2002, J. Org. Chem., 67, 1041-1044).

If the above set of conditions combination is not applicable to obtain boronic ester or acid (P5), suitable methods of preparation known by a person skilled in the art should be used.

Organotin reagents (P6) may be commercially available from various sources or synthesized, using conditions known by a person skilled in the art (Fugami et al., 2002, above). Typically, heterocycle halide, (P10), may be first transformed into the corresponding organolithium reagent (P14) by halogen metal exchange reaction with nBuLi at low temperature. The resulting organolithium reagent (P14) may further react with trialkyltinchloride ClSn(R¹⁰)₃, where R¹⁰ is methyl or n-butyl, to afford the corresponding organotin reagent (P6) (e.g. Zhang et al., 2004, J. Med. Chem. 47, 2453-2465).

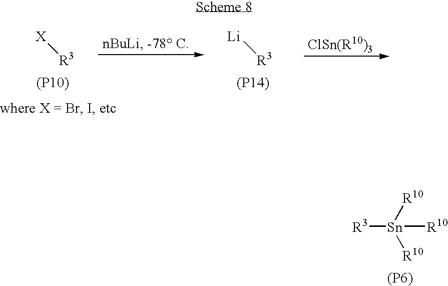

Compounds of formula (P4), with X=Br or I, may be transformed into the corresponding boronic acid or ester (P15), where R⁹ may be H, for boronic acid derivatives, or any alkyl or substituted alkyl groups for boronic ester derivatives, including optionally —B(OR⁹)₂ forming a cycle such as boronic acid pinacol ester, or the corresponding tin reagent (P16), where R¹⁰ is methyl or n-butyl, following procedures described above for the preparation of (P5) and (P6) or alternative procedures known by a person skilled in the art (Scheme 9, below). Compounds of Formula (I) may be then obtained by metal catalysed cross-coupling reaction. For instance, they may be obtained by Suzuki coupling reaction between aryl halide (P10), where X may be Br, I etc, and the boronic acid or ester (P15) (Scheme 9, below) (Bellina et al., 2004, above). In the other hand, Stille coupling may be used for the preparation of compounds of Formula (I), involving the reaction between aryl halide (P 10), where X may be Br, I etc, and the tin reagent (P16) (Scheme 9, below) (Fugami et al., 2002, above).

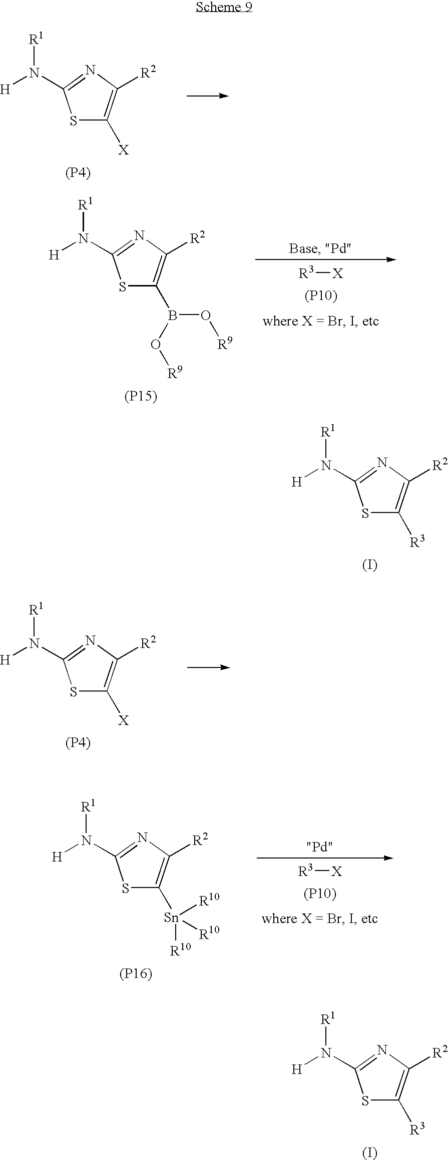

Compounds of formula (P4) with X═Br or I may be prepared by halogenation of the corresponding thiazole (P17) with reagents such as Br₂, I₂ or NBS, NIS (Scheme 10, below). Depending on the nature of R¹, protection of the secondary amine may be needed before the halogenation, with for example PG=acetyl or any other group which is easily removable.

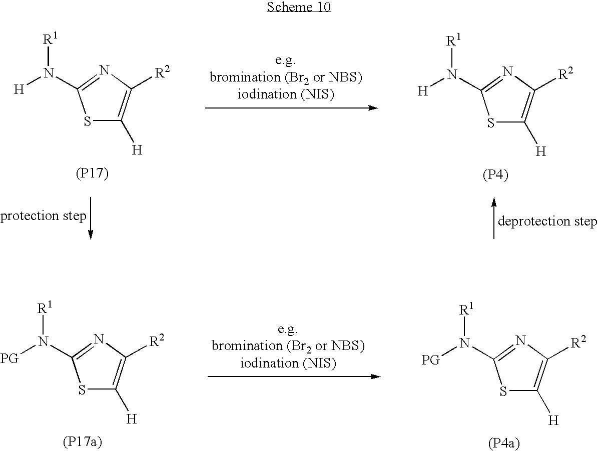

Thiazole (P17) may be commercially available from various sources or synthesized, using conditions known by a person skilled in the art, using both solution-phase and solid-phase chemistry protocols (Kodomari et al., 2002, Tetrahedron Lett., 43, 1717-1720). For example, it may be obtained in two steps (Scheme 11 below), starting with α-halogenation of a ketone (P18), using for example Br₂ for a bromination or thionyl chloride for a chlorination, affording an intermediate (P19). “Hal” in intermediate (P19) can be also a tosyloxy group, which may be introduced with suitable reagents such as hydroxy(tosyloxy)iodobenzene. Intermediate (P19) may be then added to a solution of a substituted thiourea R¹NHC(S)NH₂ (P20) in a suitable solvent, preferably a polar solvent, e.g. EtOH, leading to intermediate (P17). The resulting intermediate (P19) may react with thiourea, affording thiazole (P21) which may be further substituted with R¹, as defined above, using conditions known by a person skilled in the art.

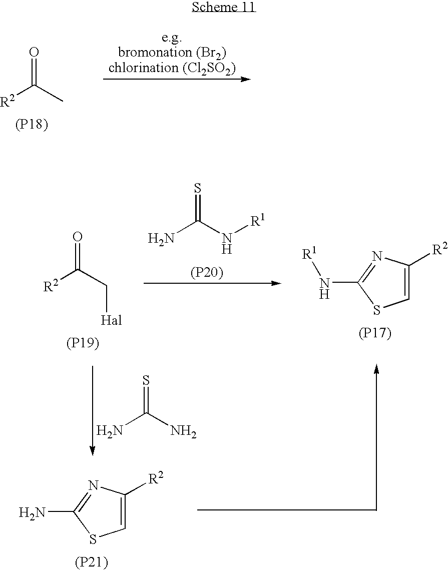

Thioureas (P20) used in synthetic Scheme 11 above are either commercially available from various sources or synthesized using conditions known by the person skilled in the art. For example, thioureas (P20) can be obtained by coupling a salt of an amine R¹NH₂, preferably HCl salt, with potassium thiocyanate used in equimolarity in THF under reflux as shown on Scheme 12 below, Pathway A.

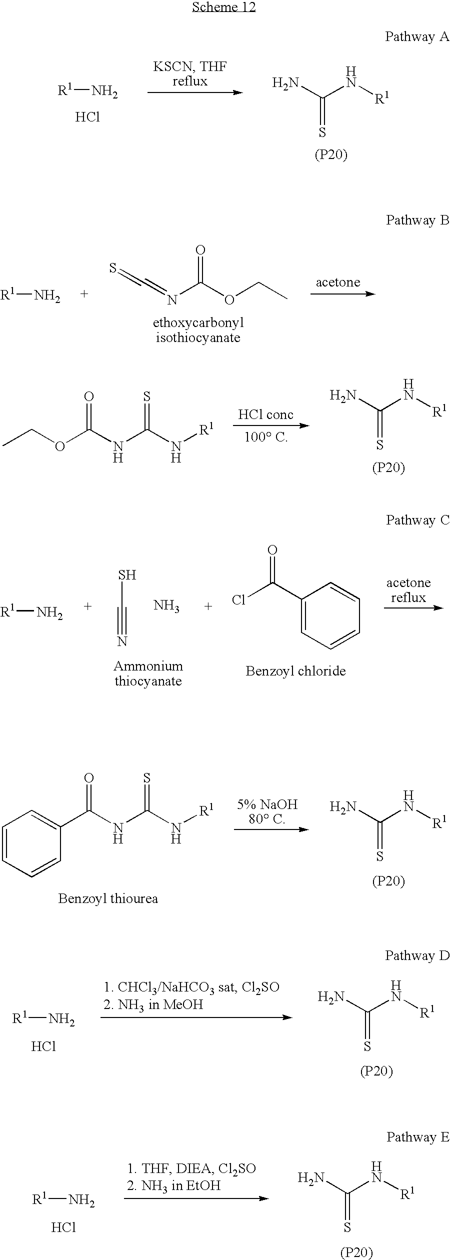

The amine R¹NH₂ can be first activated with ethoxycarbonyl isothiocyanate affording an ethoxycarbonyl thiourea intermediate, as presented above on Scheme 12 above, Pathway B. The desired thiourea (P20) is released, upon deprotection under acidic conditions, e.g. concentrated HCl. The amine R¹NH₂ can be also activated with benzoyl isothiocyanate, which is obtained by addition of benzoyl chloride to ammonium thiocyanate, giving a benzoyl thiourea intermediate, as shown above on Scheme 12 above, Pathway C. Upon deprotection under basic conditions, e.g. NaOH, the desired thiourea (P20) is released. Alternatively, the amine R¹NH₂ can react with thiophosgene, followed by the addition of ammonia, as presented above on Scheme 12 above, Pathway D. If the above set of synthetic methods is not applicable to obtain N-substituted thiourea (P20), suitable methods of preparation known by a person skilled in the art should be used.

### Methods for Preparing Intermediates of Compounds of Formula (I).

According to a further general process, compounds of Formula (I) can be converted to alternative compounds of Formula (I), employing suitable interconversion techniques well known by a person skilled in the art.

If the above set of general synthetic methods is not applicable to obtain compounds according to Formula (I) and/or necessary intermediates for the synthesis of compounds of Formula (I), suitable methods of preparation known by a person skilled in the art should be used. In general, the synthesis pathways for any individual compound of Formula (I) will depend on the specific substitutents of each molecule and upon the ready availability of intermediates necessary; again such factors being appreciated by those of ordinary skill in the art. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Compounds of this invention can be isolated in association with solvent molecules by crystallization from evaporation of an appropriate solvent. The pharmaceutically acceptable acid addition salts of the compounds of Formula (I), which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of Formula (I) with a suitable base. Both types of salts may be formed or interconverted using ion-exchange resin techniques.

In the following the present invention shall be illustrated by means of some examples, which are not construed to be viewed as limiting the scope of the invention.

### EXAMPLES

The following starting materials commercially available were used:

2-acetamido-4-methylthiazole (Aldrich), N-iodosuccinimide (Aldrich), methanesulfonyl chloride (Aldrich), 4-(4,4,5,5-tetramethyl-[1,3,2]dioxaborolan-2-yl)-1H-pyrazole (Boron-Mol), 1-methyl-4-(4,4,5,5-tetramethyl-1,3,2-dioxaborolan-2-yl)-1H pyrazole (Boron-Mol), 4-(4,4,5,5-tetramethyl-1,3,2-dioxaborolan-2-yl)-pyrazole-1-carboxylic acid tert-butyl ester (Boron-Mol), 1-benzyl-4-(4,4,5,5-tetramethyl-1,3,2-dioxaborolan-2-yl)-1H-pyrazole (Boron-Mol), 2-dicyclohexylphosphino-2′,6′-dimethoxy-1,1′-biphenyl (Aldrich), palladium(II) acetate (Acros), potassium fluoride (Fluka), 1,1′-carbonyldiimidazole (Fluka), 2-dimethylaminoethylamine (Fluka), 4-methyl-1-piperazinecarbonyl chloride (Aldrich), 6-methoxy-pyridine-3-sulfonyl chloride (Anichem), 6-chloro pyridine-3-sulfonyl chloride (Anichem), 6-morpholin-4-yl-pyridine-3-sulfonyl chloride (Maybridge), 3,5-dimethylisoxazole-4-sulfonyl chloride (ABCR), 5-chloro-1,3-dimethylpyrazole-4-sulphonyl chloride (Maybridge), 1,2-dimethylimidazole-5-sulphonyl chloride (Apollo), 1-methyl-1H-imidazole-4-sulfonyl chloride (Maybridge), 4-(chlorosulfonyl)benzoic acid (Aldrich), 3-(chlorosulfonyl)benzoic acid (Aldrich), N-Cbz-4-piperidine sulfonyl chloride (Magical), isopropylsulfonyl chloride (Aldrich), tributyltin chloride (Fluka), 2-Bromo-5-methanesulfonyl-pyridine (Acmeca), 1,1′-bis(diphenylphosphino)ferrocenedichloro palladium(II) (Avocado).

The HPLC, NMR and MS data provided in the examples described below are obtained as followed: HPLC: column Waters Symmetry C8 50×4.6 mm, Conditions: MeCN/H₂O, 5 to 100% (8 min), max plot 230-400 nm; Mass spectra: PE-SCIEX API 150 EX (APCI and ESI), LC/MS spectra: Waters ZMD (ES); ¹H-NMR: Bruker DPX-300 MHz.

The preparative HPLC purifications are performed with HPLC Waters Prep LC 4000 System equipped with columns Prep Nova-Pak HR C186 μm 60 Å, 40×30 mm (up to 100 mg) or with XTerra® Prep MS C8, 10 μm, 50×300 mm (up to Ig). All the purifications are performed with a gradient of MeCN/H₂O 0.09% TFA. The semi-preparative reverse-phase HPLC are performed with the Biotage Parallex Flex System equipped with columns Supelcosil™ ABZ+Plus (25 cm×21.2 mm, 12 μm); UV detection at 254 nm and 220 nm; flow 20 ml/min (up to 50 mg). TLC Analysis is performed on Merck Precoated 60 F₂₅₄ plates. Purifications by flash chromatography are performed on SiO₂ support, using cyclohexane/EtOAc, DCM/MeOH or CHCl₃/MeOH mixtures as eluents.

The microwave chemistry is performed on a single mode microwave reactor Emrys™ Optimiser from Personal Chemistry.

### Intermediate 1: Preparation of N-(5-iodo-4-methyl-1,3-thiazol-2-yl)acetamide (Intermediate (P4) wherein R¹ is C(O)CH₃, R² is CH₃ and X is I) (General Scheme 10)

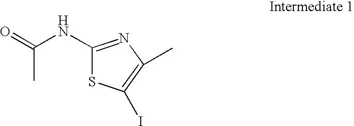

To a solution of 2-acetamido-4-methylthiazole (5 g; 32 mmol; 1 eq.) in MeCN (100 ml) was added N-iodosuccinimide (8.6 g; 38.4 mmol; 1.2 eq.). The resulting homogeneous solution was stirred at RT. After 5 min, a precipitate was formed. It was filtered and washed with cold MeCN. A first batch of Intermediate 1 was isolated as white-off solid (5 g; 57%). The mother liquors were evaporated and dissolved in EtOAc. They were washed with two fractions of Na₂S₂O₃ 1N aqueous solution and dried over MgSO₄. After filtration and evaporation of the solvents, the resulting solid was suspended in MeCN, filtered and dried under vacuo, affording a second batch of Intermediate 1 as white-off solid (1.8 g; 20%). The overall yield of this reaction was 77%. ¹H NMR (DMSO-d₆, 300 MHz) δ 1.88 (s, 3H), 2.02 (s, 3H), 12.02 (s, 1H). M⁻(ESI): 281.02; M⁺(ESI): 283.09. HPLC, Rt: 2.55 min (purity: 100%).

### Intermediate 2: Preparation of 1-(methylsulfonyl)-4-(4,4,5,5-tetramethyl-1,3,2-dioxaborolan-2-yl)-1H-pyrazole (Intermediate (P5c) wherein R³ is 1-(methylsulfonyl)-1H-pyrazol-4-yl)

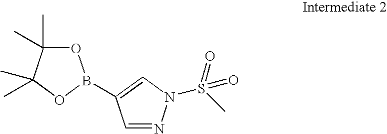

4-(4,4,5,5-tetramethyl-[1,3,2]dioxaborolan-2-yl)-1H-pyrazole (194 mg; 1 mmol; 1 eq) was dissolved in DCM (5 ml). Triethylamine (0.17 ml; 1.2 mmol; 1.2 eq.) followed by methanesulfonyl chloride (0.09 ml; 1.2 mmol; 1.2 eq.) were added. The resulting solution was stirred at RT for 1 hour. The reaction mixture was washed with water (5 ml), brine (5 ml) and dried over MgSO₄. Filtration and evaporation of the solvents afforded Intermediate 2 as an oil (229 mg; 84%). ¹H NMR (DMSO-d₆, 300 MHz) δ 1.31 (s, 12H), 3.30 (s, 3H), 8.00 (s, 1H), 8.31 (s, 1H). M⁻(ESI): 271.01; M⁺(ESI): 273.20.

### Example 1

### N-[4-methyl-5-(1-methyl-1H-pyrazol-4-yl)-1,3-thiazol-2-yl]acetamide (1)

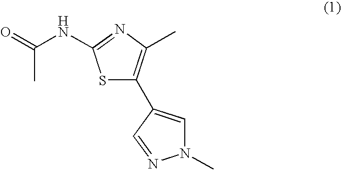

N-(5-iodo-4-methyl-1,3-thiazol-2-yl)acetamide, Intermediate 1 (141 mg; 0.5 mmol; 1 eq.), 1-methyl-4-(4,4,5,5-tetramethyl-1,3,2-dioxaborolan-2-yl)-1H pyrazole (208 mg; 1 mmol; 2.0 eq.), potassium fluoride (87 mg; 1.5 mmol; 3 eq.), 2-dicyclohexylphosphino-2′,6′-dimethoxy-1,1′-biphenyl (51 mg; 0.12 mmol; 0.25 eq.) and palladium(II) acetate (11 mg; 0.05 mmol; 0.1 eq.) were mixed in a flask kept under argon. Toluene (2.5 ml) and MeOH (2.5 ml) were added and the resulting solution was flushed with argon. The mixture was heated 6 hours at 100° C. It was filtered over a pad of celite and the solvents were evaporated. The crude product was dissolved in EtOAc, washed with NaHCO₃, water and brine. The organic phase was dried over MgSO₄, filtered and evaporated, affording a yellow crude product, which was purified by flash chromatography (CHCl₃/MeOH gradient). It was then suspended in Et₂O, filtered and washed with Et₂O, affording Compound (1) as a white-off solid (67 mg; 57%). ¹H NMR (DMSO-d₆, 300 MHz) δ 2.13 (s, 3H), 2.32 (s, 3H), 3.88 (s, 3H), 7.60 (s, 1H), 7.95 (s, 1H), 12.00 (s, 1H). M⁻(ESI): 235.28; M⁺(ESI): 237.23. HPLC, Rt: 1.89 min (purity: 97.19%).

### Example 2

### N-{4-methyl-5-[1-(methylsulfonyl)-1H-pyrazol-4-yl]-1,3-thiazol-2-yl}acetamide (2)

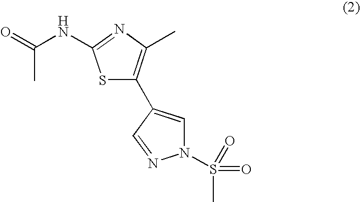

N-(5-iodo-4-methyl-1,3-thiazol-2-yl)acetamide (Intermediate 1) (120 mg; 0.4 mmol; 1. eq.), potassium fluoride (74 mg; 1.3 mmol; 3 eq.), palladium(II) acetate (9.5 mg; 0.04 mmol; 0.1 eq.), 2-dicyclohexylphosphino-2′,6′-dimethoxy-1,1′-biphenyl (17 mg; 0.04 mmol; 0.1 eq.) were mixed in a flask kept under argon. 1-(Methylsulfonyl)-4-(4,4,5,5-tetramethyl-1,3,2-dioxaborolan-2-yl)-1H-pyrazole (Intermediate 2) (174 mg; 0.64 mmol; 1.5 eq.), was added as solution in toluene (2.0 ml), followed by MeOH (2.0 ml) and water (20 μl). The resulting mixture was flushed with argon and stirred at 55° C. for 4 hours. Solvents were evaporated and the crude product was dissolved in EtOAc, washed with NaHCO₃, water and brine. Organic phase was dried over MgSO₄, filtered and evaporated. The resulting crude product was purified by preparative HPLC, affording compound (2) as white-off solid (98 mg; 77%). ¹H NMR (DMSO-d⁶) δ 2.13 (s, 3H); 2.35 (s, 3H); 3.59 (s, 3H); 8.18 (s, 1H); 8.41 (s, 1H); 12.13 (s, 1H). M⁻(ESI): 299.12; M⁺(ESI): 301.20. HPLC, Rt: 2.17 min (purity: 100%).

### Example 3

### N-[5-(1-benzyl-1H-pyrazol-4-yl)-4-methyl-1,3-thiazol-2-yl]acetamide (3)

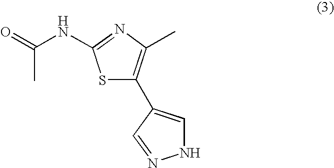

N-(5-iodo-4-methyl-1,3-thiazol-2-yl)acetamide (Intermediate 1) (282 mg; 1 mmol; 1 eq.), 4-(4,4,5,5-tetramethyl-1,3,2-dioxaborolan-2-yl)-pyrazole-1-carboxylic acid tert-butyl ester (441 mg; 1.5 mmol; 1.5 eq.), potassium fluoride (174 mg; 3 mmol; 3 eq.) palladium(II) acetate (22 mg; 0.1 mmol; 0.1 eq.) and 2-dicyclohexylphosphino-2′,6′-dimethoxy-1,1′-biphenyl (41 mg; 0.1 mmol; 0.1 eq.) were mixed in a flask kept under argon. Toluene (5 ml), MeOH (5 ml) and water (11 μl) were added. The resulting mixture was flushed with argon and stirred at 70° C. overnight. Solvents were evaporated and the crude mixture was suspended in EtOAc. The desired product was extracted with HCl 1 N aqueous solution, which was neutralized with NaOH 5N solution. The resulting aqueous phase was extracted with 2 fractions of EtOAc. Combined organic phases were dried over Na₂SO₄, filtered and evaporated. The resulting crude yellow product was suspended in Et₂O, filtered and washed with Et₂O, affording compound (3) as a white-off solid (103 mg; 46%). HPLC, Rt: 2.17 min (purity: 96%).

To prepare its HCl salt, Compound (3) was suspended in MeOH and 2 equivalents of HCl in MeOH were added (1.25 M solution). The solution was stirred for 30 min and the solvents were evaporated. The resulting powder was suspended in Et₂O and filtered, affording compound (3) as HCl salt (80 mg; 31%). ¹H NMR (DMSO-d⁶) δ 2.14 (s, 3H), 2.33 (s, 3H), 7.82 (s, 2H), 11.99 (s, 1H). M⁻(ESI): 221.3; M⁺(ESI): 223.2. HPLC, Rt: 1.55 min (purity: 99.5%).

### Example 4

### N-[5-(1-benzyl-1H-pyrazol-4-yl)-4-methyl-1,3-thiazol-2-yl]acetamide (4)

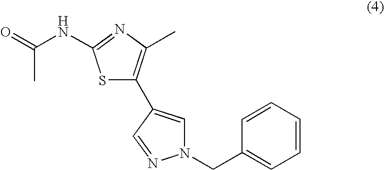

N-(5-iodo-4-methyl-1,3-thiazol-2-yl)acetamide, Intermediate 1 (282 mg; 1 mmol; 1 eq.), 1-benzyl-4-(4,4,5,5-tetramethyl-1,3,2-dioxaborolan-2-yl)-1H-pyrazole (426 mg; 1.5 mmol; 1.5 eq.), potassium fluoride (174 mg; 3 mmol; 3 eq.) palladium(II) acetate (22 mg; 0.1 mmol; 0.1 eq.) and 2-dicyclohexylphosphino-2′,6′-dimethoxy-1,1′-biphenyl (41 mg; 0.1 mmol; 0.1 eq.) were mixed in a flask kept under argon. Toluene (5 ml), MeOH (5 ml) and water (11 μl) were added. The mixture was flushed with argon and stirred for 6 hours at 100° C. Solvents were evaporated and the crude product was partially dissolved in EtOAc and washed with NaHCO₃ and brine. The organic phase was not homogeneous. It was evaporated and the resulting crude product was suspended in DCM, filtered and washed with DCM, affording Compound (4) as white-off precipitate (192 mg; 61%). ¹H NMR (DMSO-d⁶) δ 2.15 (s, 3H), 2.33 (s, 3H), 5.37 (s, 2H), 7.34 (m, 5H), 7.67 (s, 1H), 8.14 (s, 1H), 12.04 (br s, 1H). M⁻(ESI): 311.3; M⁺(ESI): 313.3. HPLC, Rt: 3.02 min (purity: 100%).

### Example 5

### 4-[2-(acetylamino)-4-methyl-1,3-thiazol-5-yl]-N-[2-(dimethylamino) ethyl]-1H-pyrazole-1-carboxamide (5)

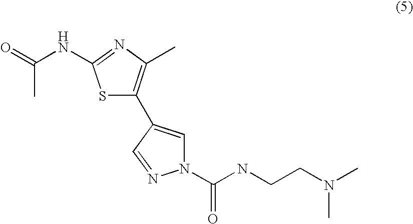

N-[4-methyl-5-(1H-pyrazol-4-yl)-1,3-thiazol-2-yl]acetamide, compound (3) (70 mg; 0.3 mmol; 1 eq.), was dissolved in DCM (6.5 ml) and DMF (0.5 ml) mixture. 1,1′-Carbonyldiimidazole (102 mg; 0.63 mmol; 2 eq.) and triethylamine (96 μl; 0.69 mmol; 2.2 eq.) were added. The reaction mixture was heated overnight at 45° C. By lowering the temperature, a precipitate was formed. It was filtered and washed with diethyl ether affording N-{5-[1-(1H-imidazol-1-ylcarbonyl)-1H-pyrazol-4-yl]-4-methyl-1,3-thiazol-2-yl}acetamide (57 mg; 58%). This intermediate was used in the next step without further purification. N-{5-[1-(1H-imidazol-1-ylcarbonyl)-1H-pyrazol-4-yl]-4-methyl-1,3-thiazol-2-yl}acetamide, obtained in the previous step (57 mg; 0.18 mmol; 1 eq.), was dissolved in DMF (3.0 ml). 2-Dimethylaminoethylamine (24 μl; 0.22 mmol; 1.2 eq.) and trimethylamine (51 μl; 0.3 mmol; 2 eq.) were added and the mixture was stirred for 10 min at RT. Solvents were evaporated. The resulting crude product was dissolved in EtOAc, extracted with NH₄Cl sat. (3 fractions). Aqueous phases were neutralised with NaOH 5N and extracted with EtOAc (3 fractions). Combined organic phases was dried over Na₂SO₄, filtered and evaporated, affording compound (5) as white-off solid (8 mg; 13%). ¹H NMR (DMSO-d⁶) δ 2.21 (s, 3H), 2.34 (s, 6H), 2.37 (s, 3H), 2.62 (t, J=6 Hz, 2H), 3.54 (t, J=6 Hz, 2H), 7.05 (br s, 1H), 7.82 (s, 1H), 8.32 (s, 1H). M⁻(ESI): 335.35; M⁺(ESI): 337.36. HPLC, Rt: 1.51 min (purity: 99%).

### Example 6

### N-(4-methyl-5-{1-[(4-methylpiperazin-1-yl)carbonyl]-1H-pyrazol-4-yl}-1,3-thiazol-2-yl)acetamide (6)

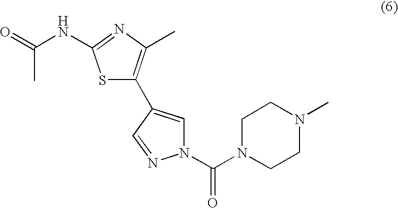

A mixture of N-[4-methyl-5-(1H-pyrazol-4-yl)-1,3-thiazol-2-yl]acetamide, Compound (3) (55 mg; 0.25 mmol; 1 eq.), 1,8-diazabicyclo[5.4.0]undec-7-ene (1, 5-5) (60 μl; 0.40 mmol; 1.6 eq.) and 4-methyl-1-piperazinecarbonyl chloride (40 mg; 0.25 mmol; 1 eq.), in THF (2.5 ml) and DMF (1.0 ml), was stirred for 24 hours at RT. The reaction mixture was partitioned between EtOAc and water. The organic layer was separated, washed with brine, dried over MgSO₄ and concentrated. The crude product was suspended in Et₂O and DCM mixture and filtered, affording compound (6) as light yellow powder (8 mg; 10%). The mother liquors were evaporated and purified by FC(CHCl₃/MeOH gradient from 20:1 to 10:1). A second batch of compound (6) was isolated (28 mg; 34%). Compound (6) was isolated with an overall yield of 44%. ¹H NMR (DMSO-d⁶) δ 2.15 (s, 3H), 2.23 (s, 3H), 2.35 (s, 3H), 2.42 (m, 4H), 3.74 (m, 4H), 8.00 (s, 1H), 8.36 (s, 1H), 12.11 (s, 1H). M⁻(ESI): 347.3; M⁺(ESI): 349.3. HPLC, Rt: 1.49 min (purity: 99.59%).

### Example 7

### N-(5-{1-[(6-methoxypyridin-3-yl)sulfonyl]-1H-pyrazol-4-yl}-4-methyl-1,3-thiazol-2-yl)acetamide (7)

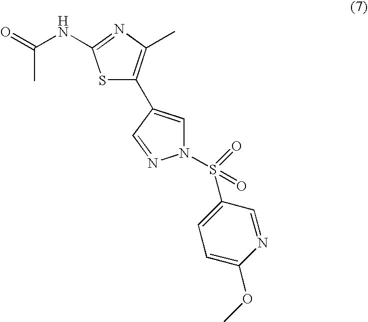

N-[4-methyl-5-(1H-pyrazol-4-yl)-1,3-thiazol-2-yl]acetamide, compound (3) (55 mg; 0.25 mmol; 1 eq.) was dissolved in DCM (2.5 ml) and DMF (1 ml). Triethylamine (0.10 ml; 0.75 mmol; 3 eq.) followed by 6-methoxy-pyridine-3-sulfonyl chloride (51 mg; 0.25 mmol; 1 eq.) were added. The mixture was stirred overnight at RT. NaHCO₃ was added and the product was extracted with three fractions of DCM. Combined organic phases was dried over MgSO₄, filtered and evaporated. The crude yellow solid was purified by flash chromatography (EtOAc/Cy 1:1), affording compound (7) as white-off solid (29 mg; 30%). ¹H NMR (DMSO-d⁶) δ 2.15 (s, 3H), 2.36 (s, 3H), 3.98 (s, 3H), 7.10 (d, J=9 Hz, 1H), 8.17 (s, 1H), 8.28 (dd, J=3.9 Hz, 1H), 8.64 (s, 1H), 8.89 (d, J=3 Hz, 1H), 12.14 (br s, 1H). M⁻(ESI): 392.3; M⁺(ESI): 394.3. HPLC, Rt: 3.26 min (purity: 92%).

### Example 8

### N-(4-methyl-5-{1-[(1-methyl-1H-imidazol-4-yl)sulfonyl]-1H-pyrazol-4-yl}-1,3-thiazol-2-yl)acetamide (8)

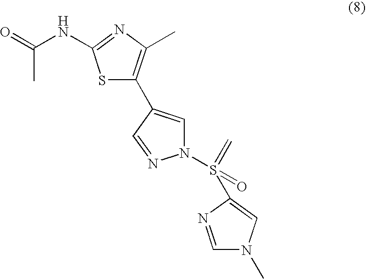

A solution of N-[4-methyl-5-(1H-pyrazol-4-yl)-1,3-thiazol-2-yl]acetamide, compound (3) (44 mg; 0.2 mmol; 1 eq.), 1-methyl-1H-imidazole-4-sulfonyl chloride (36 mg; 0.2 mmol; 1 eq.) and N,N-diisopropylethylamine (0.1 ml; 0.6 mmol; 3 eq.) in DCM (2 ml) was stirred overnight at RT. The reaction mixture was diluted with DCM and washed with water and brine. Organic phase was dried over MgSO₄, filtered and evaporated. The resulting crude product was purified by flash chromatography (CHCl₃/MeOH gradient from 100:1 to 50: 50), affording compound (8) as light yellow solid (35 mg; 47.5%). ¹H NMR (DMSO-d⁶) δ 2.12 (s, 3H), 2.32 (s, 3H), 3.72 (s, 3H), 7.86 (m, 1H), 8.06 (s, 1H), 8.30 (m, 1H), 8.47 (s, 1H), 12.11 (br s, 1H). M⁻(ESI): 365.25; M⁺(ESI): 367.23. HPLC, Rt: 2.31 min (purity: 98.4%).

### Example 9

### N-(5-{1-[(6-chloropyridin-3-yl)sulfonyl]-1H-pyrazol-4-yl}-4-methyl-1,3-thiazol-2-yl)acetamide (9)

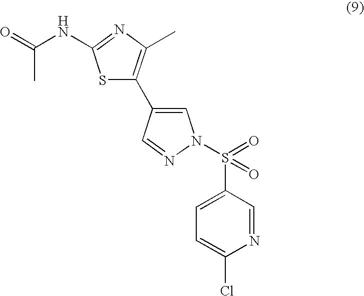

N-[4-methyl-5-(1H-pyrazol-4-yl)-1,3-thiazol-2-yl]acetamide, compound (3) (150 mg; 0.67 mmol; 1 eq.), was suspended in DCM (3.0 ml). N,N-diisopropylethylamine (0.34 ml; 2 mmol; 3 eq.) and 6-chloro pyridine-3-sulfonyl chloride (143 mg; 0.67 mmol; 1 eq.) were added successively at 0° C. DMF (3 ml) was added to improve the solubility of the mixture. The mixture was stirred for 2 days at RT. DCM was added and the resulting precipitate was filtered and rinsed with DCM, affording compound (9) as beige powder (40 mg; 15%). The filtrate was washed with water, brine and dried over MgSO₄. After filtration and evaporation of the solvents, the crude product was crystallized in EtOAc/Cyclohexane mixture, affording a second batch of compound (9) (40 mg; 15%). Compound (9) was isolated in 30% overall yield. ¹H NMR (DMSO-d⁶) δ 2.12 (s, 3H), 2.32 (s, 3H), 7.65 (d, J=8 Hz, 1H), 8.06 (s, 1H), 8.30 (d, J=8 Hz, 1H), 8.52 (s, 1H), 8.85 (s, 1H), 12.11 (s, 1H). M⁻(ESI): 396.15; M⁺(ESI): 398.18. HPLC, Rt: 3.16 min (purity: 87%).

### Example 10

### N-(4-methyl-5-{1-[(6-morpholin-4-ylpyridin-3-yl)sulfonyl]-1H-pyrazol-4-yl}-1,3-thiazol-2-yl)acetamide (10)

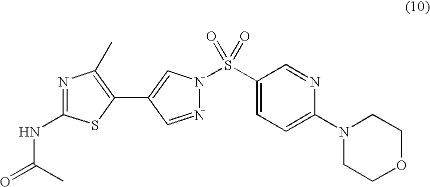

N-[4-methyl-5-(1H-pyrazol-4-yl)-1,3-thiazol-2-yl]acetamide, compound (3) (50 mg; 0.22 mmol; 1 eq.) was dissolved in pyridine (0.25 ml). 6-Morpholin-4-yl-pyridine-3-sulfonyl chloride (59 mg; 0.22 mmol; 1 eq.) was added and the resulting mixture was stirred for 1 hour at RT. As the reaction was very slow, an excess of 6-morpholin-4-yl-pyridine-3-sulfonyl chloride (295 mg; 1.1 mmol; 5 eq.) was added and the reaction mixture was stirred overnight at RT. It was diluted with DCM and washed successively with NH₄Cl sat, HCl 0.01M (twice) and 1M solution of CuSO₄. The organic phase was dried over MgSO₄ and evaporated. The resulting crude product was suspended in EtOAc, filtered and dried under vacuo, affording compound (10) as white-off solid (27 mg; 27%). ¹H NMR (DMSO-d⁶) δ 2.12 (s, 3H), 2.32 (s, 3H), 3.65 (br s, 8H), 6.95 (d, J=8 Hz, 1H), 7.95 (d, J=8 Hz, 1H), 8.06 (s, 1H), 8.52 (s, 1H), 8.65 (s, 1H), 12.11 (s, 1H). M⁻(ESI): 447.3; M⁺(ESI): 449.4. HPLC, Rt: 3.13 min (purity: 95%).

### Example 11

### N-(5-{1-[(3,5-dimethylisoxazol-4-yl)sulfonyl]-1H-pyrazol-4-yl}-4-methyl-1,3-thiazol-2-yl)acetamide (11)

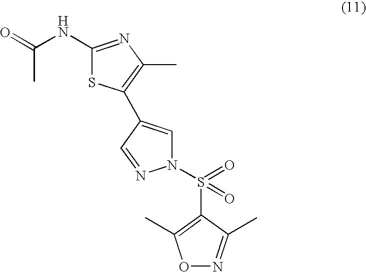

N-[4-methyl-5-(1H-pyrazol-4-yl)-1,3-thiazol-2-yl]acetamide, compound (3) (50 mg; 0.22 mmol; 1 eq.), was dissolved in pyridine (2 ml). 3,5-Dimethylisoxazole-4-sulfonyl chloride (220 mg; 1.1 mmol; 5 eq.) was added and the mixture was stirred for 2 hours at RT and 1 hour at 60° C. It was diluted with DCM and washed successively with HCl 0.1 M (3 times), CuSO₄ 1 M solutions and water. The organic phase was dried over MgSO₄ and evaporated. The crude product was suspended in EtOAc/Cyclohexane 1:1 mixture, filtered and dried under vacuo, affording compound (11) as yellow powder (27 mg; 31%). ¹H NMR (DMSO-d⁶) δ 2.12 (s, 3H), 2.30 (s, 3H), 2.32 (s, 3H), 3.72 (s, 3H), 8.15 (s, 1H), 8.70 (s, 1H), 12.11 (s, 1H). M⁻(ESI): 380.2; M⁺(ESI): 382.2. HPLC, Rt: 3.50 min (purity: 98%).

### Example 12

### N-(5-{1-[(5-chloro-1,3-dimethyl-1H-pyrazol-4-yl)sulfonyl]-1H-pyrazol-4-yl}-4-methyl-1,3-thiazol-2-yl)acetamide (12)

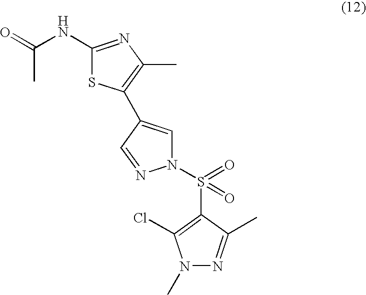

N-[4-methyl-5-(1H-pyrazol-4-yl)-1,3-thiazol-2-yl]acetamide, Compound (3) (50 mg; 0.22 mmol; 1 eq.) was dissolved in pyridine (2 ml). 5-Chloro-1,3-dimethylpyrazole-4-sulphonyl chloride (257 mg; 1.1 mmol; 5 eq.) was added and the resulting mixture was stirred for 2 hours at RT. It was diluted with DCM and washed successively with HCl 0.1 M (3 times), CuSO₄ 1 M solutions and water. The organic phase was dried over MgSO₄ and evaporated. The crude product was suspended in EtOAc/Cyclohexane 1:1 mixture, filtered and dried under vacuo, affording compound (11) as yellow powder (27 mg; 29%). M⁻(ESI): 413.2; M⁺(ESI): 415.2. HPLC, Rt: 3.11 min (purity: 86%).

### Example 13

### N-(5-{1-[(1,2-dimethyl-1H-imidazol-5-yl)sulfonyl]-1H-pyrazol-4-yl}-4-methyl-1,3-thiazol-2-yl)acetamide (13)

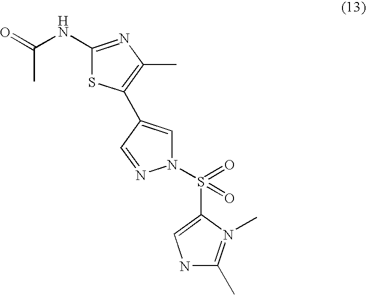

N-[4-methyl-5-(1H-pyrazol-4-yl)-1,3-thiazol-2-yl]acetamide, compound (3) (50 mg; 0.22 mmol; 1 eq.) was dissolved in pyridine (2 ml). 1,2-Dimethylimidazole-5-sulphonyl chloride (218 mg; 1.1 mmol; 5 eq.) was added and the resulting mixture was stirred for 2 hours at RT and 1 hour at 60° C. It was diluted with DCM and washed successively with HCl 0.1 M (3 times), CuSO₄ 1 M solutions and water. The organic phase was dried over MgSO₄ and evaporated. The crude product was suspended in EtOAc 1:1 mixture, filtered and dried under vacuo, affording compound (11) as yellow powder (28 mg; 33%). M⁻(ESI): 379.17; M⁺(ESI): 381.16. HPLC, Rt: 2.17 min (purity: 97%).

### Example 14

### 4-({4-[2-(acetylamino)-4-methyl-1,3-thiazol-5-yl]-1H-pyrazol-1-yl}sulfonyl)benzoic acid (14)

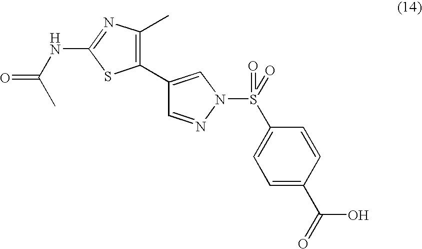

N-[4-methyl-5-(1H-pyrazol-4-yl)-1,3-thiazol-2-yl]acetamide, Compound (3) (50 mg; 0.22 mmol; 1 eq.) was suspended in DCM (2.5 ml). N,N-diisopropylethylamine (116 μl; 0.67 mmol; 3 eq.) and 4-(chlorosulfonyl)benzoic acid (49 mg; 0.22 mmol; 1 eq.) were added. The resulting yellow suspension was stirred for 3 hours at RT. HCl 1 N in Et₂O (2 eq., 0.44 ml) was added. The resulting precipitate was filtered and washed with hot EtOH, affording compound (14) as white-off solid (5 mg; 5%). M⁻(ESI): 405.2; M⁺(ESI): 407.2. HPLC, Rt: 2.02 min (purity: 69%).

### Example 15

### 3-({4-[2-(acetylamino)-4-methyl-1,3-thiazol-5-yl]-1H-pyrazol-1-yl}sulfonyl)benzoic acid (15)

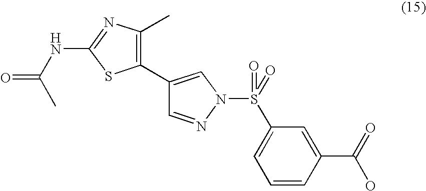

N-[4-methyl-5-(1H-pyrazol-4-yl)-1,3-thiazol-2-yl]acetamide, compound (3) (100 mg; 0.45 mmol; 1 eq.) was suspended in DCM (2.5 ml). N,N-diisopropylethylamine (231 μl; 1.3 mmol; 3 eq.) and 3-(chlorosulfonyl)benzoic acid (99 mg; 0.45 mmol; 1 eq.) were added and the reaction mixture was stirred for 2 h at RT. A second batch of 3-(chlorosulfonyl)benzoic acid (49 mg; 0.22 mmol; 0.5 eq.) was added and the mixture was stirred overnight. After one night, it became homogeneous. HCl solution in MeOH (2N, 5 eq.) was added and a precipitate was formed. It was filtered and washed with hot EtOH, affording compound (15) as yellow solid (11 mg; 6%). M⁻(ESI): 405.2; M⁺(ESI): 407.1. HPLC, Rt: 1.83 min (purity: 63.4%).

### Example 16

### benzyl 4-({4-[2-(acetylamino)-4-methyl-1,3-thiazol-5-yl]-1H-pyrazol-1-yl}sulfonyl)piperidine-1-carboxylate (16)

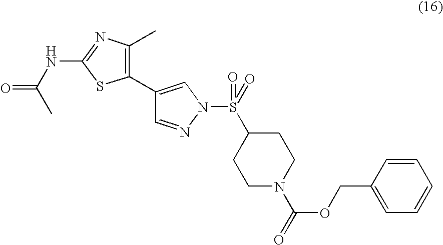

N-[4-methyl-5-(1H-pyrazol-4-yl)-1,3-thiazol-2-yl]acetamide, compound (3) (150 mg; 0.67 mmol; 1 eq.) was dissolved in pyridine (3 ml). N-Cbz-4-piperidine sulfonyl chloride (1072.3 mg; 3.3 mmol; 5 eq.) was added and the mixture was stirred overnight at RT. The solvents were evaporated and the resulting black oil was purified by preparative HPLC, affording compound (16) as white-off powder (48 mg; 14%). ¹H NMR (DMSO-d⁶) δ 2.14 (s, 3H), 2.36 (s, 3H), 2.52 (m, 2H), 2.75 (m, 2H), 3.70 (m, 2H), 4.13 (m, 2H), 5.15 (s, 2H), 6.26 (m, 1H), 7.34-7.41 (m, 5H), 7.83 (s, 1H), 8.30 (s, 1H), 12.06 (s, 1H). M⁻(ESI): 502.05; M⁺(ESI): 504.28. HPLC, Rt: 3.78 min (purity: 92%).

### Example 17

### N-{5-[1-(isopropylsulfonyl)-1H-pyrazol-4-yl]-4-methyl-1,3-thiazol-2-yl}acetamide (17)

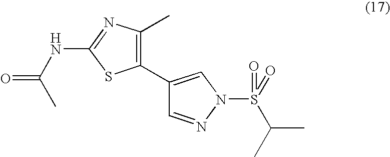

N-[4-methyl-5-(1H-pyrazol-4-yl)-1,3-thiazol-2-yl]acetamide, Compound (3) (100 mg; 0.45 mmol; 1 eq.) was dissolved in pyridine (2 ml). Isopropylsulfonyl chloride (0.25 ml; 2.25 mmol; 5 eq.) was added and the reaction mixture was stirred overnight at RT. The solvents were evaporated and the resulting black oil was purified by preparative HPLC, affording compound (17) as colourless powder (39 mg; 26%). ¹H NMR (DMSO-d⁶) δ 1.05 (d, J=6.78 Hz, 6H), 1.95 (s, 3H), 2.16 (s, 3H), 3.74 (sept, J=6.78 Hz, 1H), 8.02 (s, 1H), 8.27 (m, 1H), 11.95 (s, 1H). M⁻(ESI): 327.04; M⁺(ESI): 329.10. HPLC, Rt: 2.82 min (purity: 96%).

### Example 18

### Tert-butyl N-[({4-methyl-5-[1-(methylsulfonyl)-1H-pyrazol-4-yl]-1,3-thiazol-2-yl}amino)carbonyl]-beta-alaninate (18)

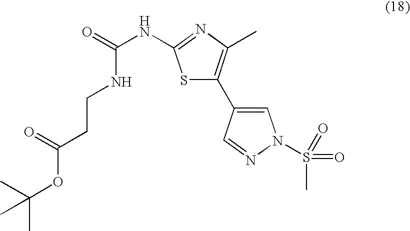

N-[4-methyl-5-(1H-pyrazol-4-yl)-1,3-thiazol-2-yl]acetamide, compound (3) (500 mg; 2.25 mmol; 1 eq.), was heated for 4 hours at 90° C. in a mixture of fuming hydrochloric acid fuming 37% (18 ml; 1.25 M; 22.5 mmol; 10 eq.) and ethanol (18 ml). The reaction mixture was cooled down to room temperature and concentrated under reduced pressure. 4-Methyl-5-(1H-pyrazol-4-yl)-thiazol-2-ylamine was precipitated in diethyl ether (20 ml) as a bis HCl salt (370 mg; 65%). ¹H NMR (DMSO-d⁶) δ 2.25 (s, 3H), 7.05 (br m, 2H), 7.80 (s, 1H), 8.35 (s, 1H). M⁻(ESI): 179.2; M⁺(ESI): 181.3. HPLC, Rt: 0.83 min (purity: 81.5%).

4-Methyl-5-(1H-pyrazol-4-yl)-1,3-thiazol-2-amine bis HCl salt (400 mg; 1.58 mmol; 1 eq.), obtained as described above, was suspended in a mixture of DCM (5 ml) and DMF (10 ml) at room temperature. Upon addition of triethylamine (1.75 ml; 12 mmol; 8 eq.), the mixture became homogeneous giving a yellow solution. The reaction was cooled down to 0° C. for 15 minutes and methane sulfonyl chloride (0.11 ml; 1.4 mmol; 0.9 eq.) was added slowly over a period of 10 minutes. The reaction was stirred at room temperature for 20 minutes and quenched with water (5 ml). The organic solvents were removed under reduced pressure and the corresponding residue was taken up in a mixture of water (5 ml) and ethyl acetate (30 ml). Organic phase was separated, dried over magnesium sulfate and evaporated to dryness. The crude product was crystallized in DCM/Et₂O (5/25) mixture, affording 5-(1-methanesulfonyl-1H-pyrazol-4-yl)-4-methyl-thiazol-2-ylamine as a white/yellow powder (405 mg, 50%). ¹H NMR (DMSO-d⁶) δ 2.18 (s, 3H), 3.34 (s, 3H), 7.15 (br m, 2H), 8.05 (s, 1H), 8.22 (s, 1H). M⁻(ESI): 257.3; M⁺(ESI): 259.3. HPLC, Rt: 1.31 min (purity: 80

4-Methyl-5-[1-(methylsulfonyl)-1H-pyrazol-4-yl]-1,3-thiazol-2-amine (250 mg; 0.97 mmol; 1 eq.), obtained as described above, was dissolved in a mixture of DCM (15 ml) and DMF (15 ml) in presence of triethylamine (0.47 ml; 3.4 mmol; 3.5 eq.). 1,1′-Carbonyldiimidazole (282 mg; 1.74 mmol; 1.8 eq.) was added and the reaction was heated at 45° C. for one night. It was cooled down to room temperature. Beta-alanine tert butyl ester hydrochloride salt (185 mg; 1.02 mmol; 1 eq.) and triethylamine (0.28 ml; 2 mmol; 2 eq.) were added and the reaction mixture was stirred at room temperature for 12 hours. The reaction was quenched with water (5 ml) and the solvents were concentrated. The residue was diluted with EtOAc, washed with water and dried over MgSO₄. After filtration and evaporation of the solvents, the resulting crude product was purified by flash chromatography using cyclohexane/ethylacetate (30/70) as eluent, affording compound (18) as white powder (35 mg; 8%). ¹H NMR (DMSO-d⁶) δ 1.44 (s, 9H), 2.37 (s, 3H), 2.56 (t, 2H, J=9 Hz), 3.39 (s, 3H), 3.60 (t, 2H, J=9 Hz), 7.88 (s, 1H), 8.05 (s, 1H). M⁻(ESI): 257.3; M⁻(ESI): 428.1; M⁺(ESI): 430.2. HPLC, Rt: 3.42 min (purity: 89%).

### Example 19

### N-{4-methyl-5-[5-(methylsulfonyl)pyridin-2-yl]-1,3-thiazol-2-yl}acetamide (19)

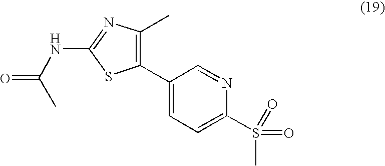

N-(5-iodo-4-methyl-1,3-thiazol-2-yl)acetamide, Intermediate 1 (282 mg; 1 mmol; 1 eq.), was dissolved in THF (10 ml). The resulting solution was cooled down to −78° C. n-Butyllithium (1.00 ml; 2 M) solution was added dropwise. After 2 hours, tributyltin chloride (358 mg; 1.1 mmol; 1.1 eq.) was added and the mixture was stirred for one hour at −78° C. and 5 hours at RT. NH₄Cl sat was added and the desired product was extracted with EtOAc (3 fractions). Combined organic phases was dried over MgSO₄, filtered and evaporated. By ¹H NMR, about 12% of the desired compound, N-[4-methyl-5-(tributylstannyl)-1,3-thiazol-2-yl]acetamide, was formed, contaminated with N-(4-methyl-1,3-thiazol-2-yl)acetamide. The crude mixture was used as such in the next step. M⁻(ESI): 445.42; M⁺(ESI): 447.50.

2-Bromo-5-methanesulfonyl-pyridine (54 mg; 0.23 mmol; 1 eq.) and 1,1′-bis(diphenylphosphino)ferrocenedichloro palladium(II) (17 mg; 0.02 mmol; 0.1 eq.) were added to a solution of N-[4-methyl-5-(tributylstannyl)-1,3-thiazol-2-yl]acetamide, obtained as a mixture in the previous step (about 0.2 mmol), in DMF (2 ml). The mixture was flushed with argon and heated 2 hours at 100° C. The reaction was complete. Solvents were evaporated and the crude mixture was dissolved in EtOAc and wash with water (3 fractions) and brine (2 fractions). The organic layers were dried over MgSO₄, filtered and evaporated. The crude mixture was purified by flash chromatography (EtOAc/Cyclohexane 1:1). The major fraction was N-(4-methyl-1,3-thiazol-2-yl)acetamide, followed by the desired product, compound (19), which was isolated as white-off solid (6 mg; 7%). M⁻(ESI): 310.2; M⁺(ESI): 312.3. HPLC, Rt: 2.13 min (purity: 100%).

### Example 20

### Biological Assays

The compounds of the present invention may be subjected to the following assays:

### a) High Throughput PI3K Lipid Kinase Assay (Binding Assay):

The efficacy of compounds of the invention in inhibiting the PI3K induced-lipid phosphorylation may be tested in the following binding assay.

The assay combines the scintillation proximity assay technology (SPA, Amersham) with the capacity of neomycin (a polycationic antibiotic) to bind phospholipids with high affinity and specificity. The Scintillation Proximity Assay is based on the properties of weakly emitting isotopes (such as ³H, ¹²⁵I, ³³P). Coating SPA beads with neomycin allows the detection of phosphorylated lipid substrates after incubation with recombinant PI3K and radioactive ATP in the same well, by capturing the radioactive phospholipids to the SPA beads through their specific binding to neomycin.

To a 384 wells MTP containing 5 μl of the test compound of Formula (I) (solubilized in 6% DMSO; to yield a concentration of 100, 30, 10, 3, 1, 0.3, 0.1, 0.03, 0.01, 01 μM of the test compound), the following assay components are added. 1) 5 μl (58 ng) of Human recombinant GST-PI3Kγ (in Hepes 40 mM, pH 7.4, DTT 1 mM and ethyleneglycol 5%) 2) 10 μl of lipid micelles and 3) 10 μl of Kinase buffer ([³³P]γ-ATP 45 μM/60 nCi, MgCl₂ 30 mM, DTT 1 mM, β-Glycerophosphate 1 mM, Na₃VO₄ 100 μM, Na Cholate 0.3%, in Hepes 40 mM, pH 7.4). After incubation at room temperature for 180 minutes, with gentle agitation, the reaction is stopped by addition of 60 μl of a solution containing 100 μg of neomycin-coated PVT SPA beads in PBS containing ATP 10 mM and EDTA 5 mM. The assay is further incubated at room temperature for 60 minutes with gentle agitation to allow binding of phospholipids to neomycin-SPA beads. After precipitation of the neomycin-coated PVT SPA beads for 5 minutes at 1500×g, radioactive PtdIns(3)P is quantified by scintillation counting in a Wallac MicroBeta™ plate counter.

The values indicated in Table I below refer to the IC₅₀ (μM) with respect to PI3Kγ, i.e. the amount necessary to achieve 50% inhibition of said target. Said values show a considerable inhibitory potency of thiazole compounds with regard to PI3Kγ.

Examples of inhibitory activities for compounds of the invention are set out in Table I below.

TABLE I
IC₅₀ values of thiazole derivatives against PI3Kγ.

| | PI3Kγ |
|---|---|
| Example No | IC₅₀ (μM) |
| 1 | 1.337 |
| 3 | 0.413 |
| 5 | 1.226 |
| 7 | 0.282 |
| 8 | 0.111 |
| 14 | 0.708 |
| 19 | 1.840 |

### b) Cell Based ELISA to Monitor PI3K Inhibition:

The efficacy of compounds of the invention in inhibiting the PI3K induced Akt/PKB phosphorylation may be tested in the following cell based assay.

Measurement of Akt/PKB phosphorylation in macrophages after stimulation with Complement 5a: Raw 264: Raw 264-7 macrophages (cultured in DMEM-F12 medium containing 10% Fetal Calf serum and antibiotics) are plated at 20'000 cells/well in a 96 MTP 24 h before cell stimulation. Prior to the stimulation with 50 nM of Complement 5a during 5 minutes, Cells are serum starved for 2 h, and pretreated with inhibitors for 20 minutes. After stimulation cells are fixed in 4% formaldehyde for 20 minutes and washed 3 times in PBS containing 1% Triton X-100 (PBS/Triton). Endogenous peroxidase is blocked by a 20 minutes incubation in 0.6% H₂O₂ and 0.1% Sodium Azide in PBS/Triton and washed 3 times in PBS/Triton. Cells are then blocked by 60 minutes incubation with 10% fetal calf serum in PBS/Triton. Next, phosphorylated Akt/PKB is detected by an overnight incubation at 4° C. with primary antibody (anti phospho Serine 473 Akt IHC, Cell Signaling) diluted 800-fold in PBS/Triton, containing 5% bovine serum albumin (BSA). After 3 washes in PBS/Triton, cells are incubated for 60 minutes with a peroxidase conjugated goat-anti-rabbit secondary antibody (1/400 dilution in PBS/Triton, containing 5% BSA), washed 3 times in PBS/Triton, and 2 times in PBS and further incubated in 100 μl of luminescent substrate reagent solution (Pierce) for 2 minutes, followed by the reading (1 s/well).

The values indicated in Table II below reflect the percentage of inhibition of AKT phosphorylation as compared to basal level. Said values show a clear effect of the thiazole compounds on the activation of AKT phosphorylation in macrophages.

Examples of inhibitory activities for compounds of the invention are set out in Table II below.

TABLE II
IC₅₀ values of thiazole derivatives in Cell Assay

| | Cell Assay (P-Akt, Elisa) |
|---|---|
| Example No | IC₅₀ [nM] |
| 8 | 850 |

### Example 21

### Thioglycollate-Induced Peritoneal Cavity Cell Recruitment Model

The in vivo efficacy of compounds of the invention in inhibiting the migration of leukocytes upon intraperitoneal challenge of thioglycollate may be tested with the following assay.

### Experimental Protocol:

8-10 weeks old female C3H mice are fasted during 18 hours. 15 minutes prior the intraperitoneal injection of thioglycollate (1.5%, 40 ml/kg), the mice are treated orally with Thiazoles of Formula (I). Control mice receive CMC/Tween as vehicle (10 ml/kg). The mice are then sacrificed by CO₂ inhalation and the peritoneal cavity is washed two times with 5 ml of ice-cold PBS/1 mM EDTA. The lavages are done 4 hours or 48 hours after thioglycollate challenge to evaluate neutrophils or macrophages recruitment, respectively. The white blood cells (neutrophils, lymphocytes or macrophages) are counted using a Beckman Coulter® A^{c}T 5Diff™. Dexamethasone is used as reference drug.

### Example 22

### Preparation of a Pharmaceutical Formulation

### Formulation 1—Tablets

A compound of Formula (I) is admixed as a dry powder with a dry gelatin binder in an approximate 1:2 weight ration. A minor amount of magnesium stearate is added as a lubricant. The mixture is formed into 240-270 mg tablets (80-90 mg) of active thiazole compound per tablet) in a tablet press.

### Formulation 2—Capsules

A compound of Formula (I) is admixed as a dry powder with a starch diluent in an approximate 1:1 weight ratio. The mixture is filled into 250 mg capsules (125 mg of active thiazole compound per capsule).

### Formulation 3—Liquid

A compound of Formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 ml.

### Formulation 4—Tablets

A compound of Formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active thiazole compound) in a tablet press.

### Formulation 5—Injection

A compound of Formula (I) is dissolved in a buffered sterile saline injectable aqueous medium to a concentration of approximately 5 mg/ml.

1-28. (canceled)

29. A thiazole derivative according to Formula (I), 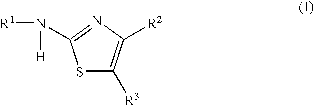
wherein
R¹ is selected from aryl, heteroaryl, C₂-C₈-cycloalkyl, heterocycloalkyl and acyl; or substituted or unsubstituted amino carbonyl;
R² is selected from H; halogen; C₁-C₆-alkyl, C₂-C₆-alkenyl or C₂-C₆-alkynyl;
R³ is selected from the following groups: 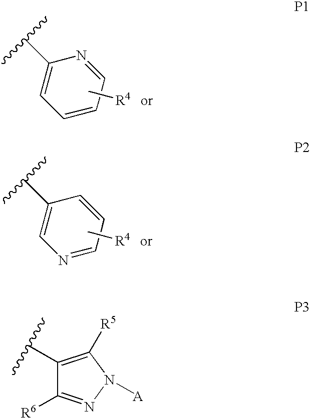
R⁴ is —SO₂—R⁸;
R⁵ and R⁶ are independently selected from H, C₁-C₆-alkyl, C₂-C₆-alkenyl, C₂-C₆-alkynyl or halogen;
R⁷ is selected from H, substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₃-C₈-cycloalkyl, substituted or unsubstituted heterocycloalkyl, amino; or substituted or unsubstituted C₁-C₆ alkyl amine;
R⁸ is selected from substituted or unsubstituted C₁-C₆-alkyl, substituted or unsubstituted C₂-C₆-alkenyl, substituted or unsubstituted C₂-C₆-alkynyl, substituted or unsubstituted aryl, substituted or unsubstituted heteroaryl, substituted or unsubstituted C₂-C₈-cycloalkyl, substituted or unsubstituted heterocycloalkyl or amino;
A is selected from H, —SO₂—R⁷, —C(O)—R⁷ substituted or unsubstituted C₁-C₆-alkyl, substituted or unsubstituted C₂-C₆-alkenyl, substituted or unsubstituted C₂-C₆-alkynyl, aryl C₁-C₆-alkyl, heteroaryl C₁-C₆-alkyl, C₂-C₈-cycloalkyl C₁-C₆-alkyl or heterocycloalkyl C₁-C₆-alkyl; and
geometrical isomers, enantiomers, diastereomers, racemates or pharmaceutically acceptable salts thereof.

30. The thiazole derivative according to claim 29, wherein R¹ is acyl.

31. The thiazole derivative according to claim 29, wherein R² is methyl.

32. The thiazole derivative according to claim 29, wherein R³ is a pyridinyl P1.

33. The thiazole derivative according to claim 29, wherein R³ is a pyridinyl P2.

34. The thiazole derivative according to claim 29, wherein R³ is a pyrazolyl P3.

35. The thiazole derivative according to claim 29, wherein R⁵ and R⁶ are H.

36. The thiazole derivative according to claim 29, wherein R⁷ is selected from H, substituted or unsubstituted C₁-C₆-alkyl, substituted or unsubstituted C₂-C₆-alkenyl, C₂-substituted or unsubstituted C₆-alkynyl or amino.

37. The thiazole derivative according to claim 29, wherein R⁷ is selected from substituted or unsubstituted aryl, substituted or unsubstituted heteroaryl, substituted or unsubstituted C₃-C₈-cycloakyl or substituted or unsubstituted C₂-C₆-heterocycloalkyl.

38. The thiazole derivative according to claim 29, wherein R⁸ is selected from substituted or unsubstituted C₁-C₆-alkyl, substituted or unsubstituted C₂-C₆-alkenyl or substituted or unsubstituted C₂-C₆-alkynyl.

39. The thiazole derivative according to claim 29, wherein R¹ is acyl; R² is methyl; and R⁵ and R⁶ are H.

40. The thiazole derivative according to claim 29, wherein said thiazole derivative is selected from:
N-[4-methyl-5-(1-methyl-1H-pyrazol-4-yl)-1,3-thiazol-2-yl]acetamide;
N-[4-methyl-5-(1-methyl-1H-pyrazol-4-yl)-1,3-thiazol-2-yl]acetamide;
N-[4-methyl-5-(1H-pyrazol-4-yl)-1,3-thiazol-2-yl]acetamide;
N-[5-(1-benzyl-1H-pyrazol-4-yl)-4-methyl-1,3-thiazol-2-yl]acetamide;
4-[2-(acetylamino)-4-methyl-1,3-thiazol-5-yl]-N-[2-(dimethylamino)ethyl]-1H-pyrazole-1-carboxamide;
N-(4-methyl-5-{1-[(4-methylpiperazin-1-yl)carbonyl]-1H-pyrazol-4-yl}-1,3-thiazol-2-yl)acetamide;
N-(5-{1-[(6-methoxypyridin-3-yl)sulfonyl]-1H-pyrazol-4-yl}-4-methyl-1,3-thiazol-2-yl)acetamide;
N-(4-methyl-5-{1-[(1-methyl-1H-imidazol-4-yl)sulfonyl]-1H-pyrazol-4-yl}-1,3-thiazol-2-yl)acetamide;
N-(5-{1-[(6-chloropyridin-3-yl)sulfonyl]-1H-pyrazol-4-yl}-4-methyl-1,3-thiazol-2-yl)acetamide;
N-(4-methyl-5-{1-[(6-morpholin-4-ylpyridin-3-yl)sulfonyl]-1H-pyrazol-4-yl}-1,3-thiazol-2-yl)acetamide;
N-(5-{1-[(3,5-dimethylisoxazol-4-yl)sulfonyl]-1H-pyrazol-4-yl}-4-methyl-1,3-thiazol-2-yl)acetamide;
N-(5-{1-[(5-chloro-1,3-dimethyl-1H-pyrazol-4-yl)sulfonyl]-1H-pyrazol-4-yl}-4-methyl-1,3-thiazol-2-yl)acetamide;
N-(5-{1-[(1,2-dimethyl-1H-imidazol-5-yl)sulfonyl]-1H-pyrazol-4-yl}-4-methyl-1,3-thiazol-2-yl)acetamide;
4-({4-[2-(acetylamino)-4-methyl-1,3-thiazol-5-yl]-1H-pyrazol-1-yl}sulfonyl)benzoic acid;
3-({4-[2-(acetylamino)-4-methyl-1,3-thiazol-5-yl]-1H-pyrazol-1-yl}sulfonyl)benzoic acid;
benzyl 4-({4-[2-(acetylamino)-4-methyl-1,3-thiazol-5-yl]-1H-pyrazol-1-yl}sulfonyl)piperidine-1-carboxylate;
N-{5-[1-(isopropylsulfonyl)-1H-pyrazol-4-yl]-4-methyl-1,3-thiazol-2-yl}acetamide; Tert-butyl N-[({4-methyl-5-[1-(methylsulfonyl)-1H-pyrazol-4-yl]-1,3-thiazol-2-yl}amino)carbonyl]-beta-alaninate; or
N-{4-methyl-5-[5-(methylsulfonyl)pyridin-2-yl]-1,3-thiazol-2-yl}acetamide.

41. A method of treating autoimmune disorders, inflammatory diseases, cardiovascular diseases, neurodegenerative diseases, bacterial or viral infections, allergy, asthma, pancreatitis, multi-organ failure, kidney diseases, platelet aggregation, cancer, transplantation, sperm motility, erythrocyte deficiency, graft rejection or lung injuries comprising the administration of a therapeutically effective amount of a compound according to claim 29 to a subject in need of treatment.

42. The method according to claim 41, wherein said inflammatory diseases are selected from multiple sclerosis, psoriasis, rheumatoid arthritis, systemic lupus erythematosus, inflammatory bowel disease, lung inflammation, thrombosis, brain infection/inflammation, meningitis or encephalitis.

43. The method according to claim 41, wherein said neurodegenerative diseases are selected from Alzheimer's disease, Huntington's disease, CNS trauma, stroke or ischemic conditions.

44. The method according to claim 41, wherein said cardiovascular diseases are selected from atherosclerosis, heart hypertrophy, cardiac myocyte dysfunction, elevated blood pressure or vasoconstriction.

45. The method according to claim 41, wherein said subject is treated for a disease selected from chronic obstructive pulmonary disease, anaphylactic shock fibrosis, psoriasis, allergic diseases, asthma, stroke or ischemic conditions, ischemia-reperfusion, platelet aggregation/activation, skeletal muscle atrophy/hypertrophy, leukocyte recruitment in cancer tissue, angiogenesis, invasion metastasis, melanoma, Kaposi's sarcoma, acute and chronic bacterial and viral infections, sepsis, graft rejection, glomerulo sclerosis, glomerulo nephritis, progressive renal fibrosis, endothelial and epithelial injuries in the lung or lung airway inflammation.

46. A pharmaceutical composition containing at least one thiazole derivative according to claim 29 and a pharmaceutically acceptable carrier, diluent or excipient thereof.

47. A process for the preparation of thiazole derivative comprising reacting a thiazole of Formula (P4) with a compound of Formula (P5) in presence of Pd and a base 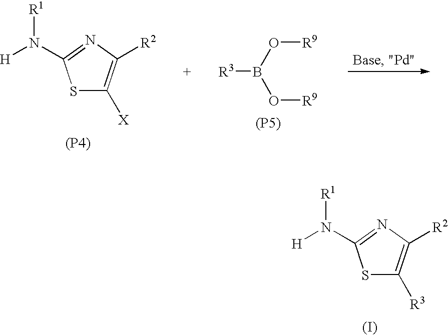
wherein R¹, R², R³ and X are as defined in claim 29 and wherein R⁹ is selected from H and C₁-C₆ alkyl.

48. A process for the preparation of thiazole derivative, comprising reacting a thiazole of Formula (P8) with a compound of Formula R⁸H in presence of a chlorination agent 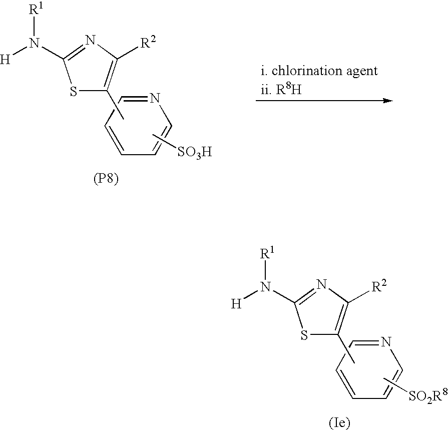
wherein R¹ and R² are as defined in claim 29 and R⁸ is an amino group.

49. A process for the preparation of thiazole derivative comprising the step of reacting a thiazole of Formula (P9) in oxidative conditions 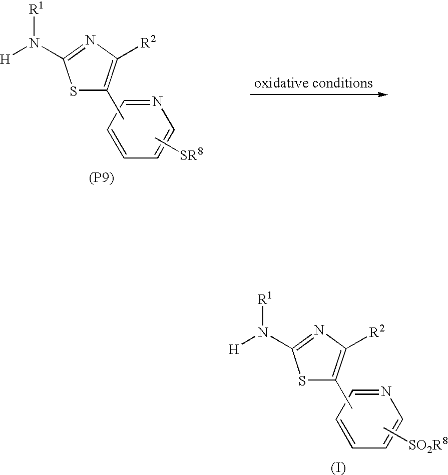
wherein R¹ and R² are as defined in claim 29 and R⁸ is selected from C₁-C₆ alkyl, C₂-C₆-alkenyl, C₂-C₆-alkynyl, aryl, heteroaryl, C₂-C₈-cycloalkyl and heterocycloalkyl.

50. A compound of:
Formula (P8): 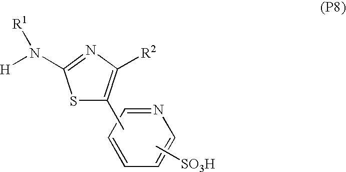
wherein R¹ and R² are as defined in claim 29; or
Formula (P9) 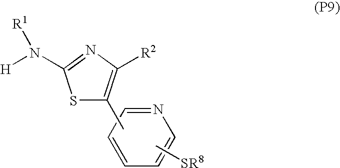
wherein R¹ and R² are as defined in claim 29 and R⁸ is selected from C₁-C₆ alkyl, C₂-C₆-alkenyl, C₂-C₆-alkynyl, aryl, heteroaryl, C₂-C₈-cycloalkyl and heterocycloalkyl.

51. The compound according to claim 50, wherein:
a) said compound is a compound of Formula (P8) and is selected from:
5-[2-(acetylamino)-4-methyl-1,3-thiazol-5-yl]pyridine-2-sulfonic acid;
6-[2-(acetylamino)-4-methyl-1,3-thiazol-5-yl]pyridine-2-sulfonic acid;
5-[2-(acetylamino)-4-methyl-1,3-thiazol-5-yl]pyridine-3-sulfonic acid;
2-[2-(acetylamino)-4-methyl-1,3-thiazol-5-yl]pyridine-4-sulfonic acid; or
6-[2-(acetylamino)-4-methyl-1,3-thiazol-5-yl]pyridine-3-sulfonic acid; or
b) said compound is a compound of Formula (P9) and is selected from:
N-{4-methyl-5-[6-(methylthio)pyridin-3-yl]-1,3-thiazol-2-yl}acetamide;
N-{4-methyl-5-[6-(methylthio)pyridin-2-yl]-1,3-thiazol-2-yl}acetamide;
N-{4-methyl-5-[5-(methylthio)pyridin-3-yl]-1,3-thiazol-2-yl}acetamide;
N-{4-methyl-5-[4-(methylthio)pyridin-2-yl]-1,3-thiazol-2-yl}acetamide; or
N-{4-methyl-5-[5-(methylthio)pyridin-2-yl]-1,3-thiazol-2-yl}acetamide.
